SEQUENCE LISTING (75 sequences)

SEQ ID NO: 1 - DNA, 33 bp, Artificial Sequence
  CCR2 molecular beacon
gcgacgcatgctggtgatcctcatcttcgtcgc

SEQ ID NO: 2 - DNA, 27 bp, Artificial Sequence
  CCR2 molecular beacon
cgcaggatgaggacgaccagcactgcg

SEQ ID NO: 3 - DNA, 28 bp, Artificial Sequence
  CCR2 molecular beacon
cgcaccatgctggtcatcctcatgtgcg

SEQ ID NO: 4 - DNA, 32 bp, Artificial Sequence
  CCR2 molecular beacon
cgcgtctgaggacgaccagcatgttggacgcg

SEQ ID NO: 5 - DNA, 37 bp, Artificial Sequence
  CCR5 molecular beacon
gcgagctcattttccatacattaaagatagtgctcgc

SEQ ID NO: 6 - DNA, 37 bp, Artificial Sequence
  CCR5 molecular beacon
cgcacgtcagtatcaattctggaagaatttccgtgcg

SEQ ID NO: 7 - DNA, 28 bp, Artificial Sequence
  SDF1 molecular beacon
cgcgtgccaggtctgcctcttctacgcg

SEQ ID NO: 8 - DNA, 25 bp, Artificial Sequence
  SDF1 molecular beacon
cgacggacccggctcccatgcgtcg

SEQ ID NO: 9 - DNA, 28 bp, Artificial Sequence
  Factor V molecular beacon
cgacgtggacaggcaaggaataccgtcg

SEQ ID NO: 10 - DNA, 27 bp, Artificial Sequence
  Factor V molecular beacon
cgacgtgtattcctcgcctgtccgtcg

SEQ ID NO: 11 - DNA, 28 bp, Artificial Sequence
  MTHFR molecular beacon
ccgcttgatgaaatcgactcccgagcgg

SEQ ID NO: 12 - DNA, 26 bp, Artificial Sequence
  MTHFR molecular beacon
ccggtgcgggagccgatttcaaccgg

SEQ ID NO: 13 - DNA, 21 bp, Artificial Sequence
  CCR2 forward PCR primer
ctctactcgctggtgttcatc

SEQ ID NO: 14 - DNA, 22 bp, Artificial Sequence
  CCR2 reverse PCR primer
gagcaggtaaatgtcagtcaag

SEQ ID NO: 15 - DNA, 22 bp, Artificial Sequence
  CCR5 forward PCR primer
cttcattacacctgcagctctc

SEQ ID NO: 16 - DNA, 19 bp, Artificial Sequence
  CCR5 reverse PCR primer
gacaagcagcggcaggacc

SEQ ID NO: 17 - DNA, 20 bp, Artificial Sequence
  CCR5 forward PCR primer
ccaggaatcatctttaccag

SEQ ID NO: 18 - DNA, 21 bp, Artificial Sequence
  CCR5 reverse PCR primer
caggaccagccccaagatgac

SEQ ID NO: 19 - DNA, 18 bp, Artificial Sequence
  SDF1 forward PCR primer
ccccttctccatccacat

SEQ ID NO: 20 - DNA, 18 bp, Artificial Sequence
  SDF1 reverse PCR primer
tgctgccctcccagaaga

SEQ ID NO: 21 - DNA, 17 bp, Artificial Sequence
  SDF1 reverse PCR primer
tgctgccctcccagaag

SEQ ID NO: 22 - DNA, 20 bp, Artificial Sequence
  Factor V forward PCR primer
gacatcatgagagacatcgc

SEQ ID NO: 23 - DNA, 23 bp, Artificial Sequence
  Factor V reverse PCR primer
aggttacttcaaggacaaaatac

SEQ ID NO: 24 - DNA, 21 bp, Artificial Sequence
  MTHFR forward PCR primer
acttgaaggagaaggtgtctg

SEQ ID NO: 25 - DNA, 22 bp, Artificial Sequence
  MTHFR reverse PCR primer
gaagaatgtgtcagcctcaaag

SEQ ID NO: 26 - DNA, 21 bp, Artificial Sequence
  MTHFR forward PCR primer
tgacctgaagcacttgaagga

SEQ ID NO: 27 - DNA, 20 bp, Artificial Sequence
  MTHFR reverse PCR primer
caaagaaaagctgcgtgatg

SEQ ID NO: 28 - DNA, 21 bp, Artificial Sequence
  Factor XIII forward PCR primer
cccaataactctaatgcagcg

SEQ ID NO: 29 - DNA, 20 bp, Artificial Sequence
  Factor XIII reverse PCR primer
tgctcataccttgcaggttg

SEQ ID NO: 30 - DNA, 28 bp, Artificial Sequence
  Factor XIII molecular beacon
cgcacgcttcagggcttggtgccgtgcg

SEQ ID NO: 31 - DNA, 28 bp, Artificial Sequence
  Factor XIII molecular beacon
gcgacgcaccacgccctgaagccgtcgc

SEQ ID NO: 32 - DNA, 20 bp, Artificial Sequence
  CCR2 forward PCR cloning primer
atgctgtccacatctcgttc

SEQ ID NO: 33 - DNA, 20 bp, Artificial Sequence
  CCR2 reverse PCR cloning primer
cccaaagacccactcatttg

SEQ ID NO: 34 - DNA, 20 bp, Artificial Sequence
  CCR5 forward PCR cloning primer
tggctgtgtttgcgtctctc

SEQ ID NO: 35 - DNA, 20 bp, Artificial Sequence
  CCR5 reverse PCR cloning primer
agataagcctcacagccctg

SEQ ID NO: 36 - DNA, 20 bp, Artificial Sequence
  SDF1 forward PCR cloning primer
cagtcaacctgggcaaagcc

SEQ ID NO: 37 - DNA, 20 bp, Artificial Sequence
  SDF1 reverse PCR cloning primer
agctttggtcctgagagtcc

SEQ ID NO: 38 - DNA, 22 bp, Artificial Sequence
  Factor V forward PCR cloning primer
tgcccagtgcttaacaagacca

SEQ ID NO: 39 - DNA, 20 bp, Artificial Sequence
  Factor V reverse PCR cloning primer
tgttatcacactggtgctaa

SEQ ID NO: 40 - DNA, 20 bp, Artificial Sequence
  Factor V reverse PCR cloning primer
cttgaacaggtggaggccag

SEQ ID NO: 41 - DNA, 20 bp, Artificial Sequence
  MTHFR reverse PCR cloning primer
aggacggtgcggtgagagtg

SEQ ID NO: 42 - DNA, 21 bp, Artificial Sequence
  Factor XIII forward PCR cloning primer
cccaataactctaatgcagcg

SEQ ID NO: 43 - DNA, 20 bp, Artificial Sequence
  Factor XIII reverse PCR cloning primer
tgctcataccttgcaggttg

SEQ ID NO: 44 - DNA, 30 bp, Artificial Sequence
  CCR2 forward for site-directed mutagenesis
gcaacatgctggtcatcctcatcttaataa

SEQ ID NO: 45 - DNA, 30 bp, Artificial Sequence
  CCR2 reverse primer for site directed mutagenesis
ttattaagatgaggatgaccagcatgttgc

SEQ ID NO: 46 - DNA, 30 bp, Artificial Sequence
  SDF1 forward site directed mutagenesis primer
tccacatgggagccaggtctgcctcttctg

SEQ ID NO: 47 - DNA, 30 bp, Artificial Sequence
  SDF1 reverse site directed mutagenesis primer
cagaagaggcagacctggctcccatgtgga

SEQ ID NO: 48 - DNA, 34 bp, Artificial Sequence
  Factor V forward site directed mutagenesis primer
gatccctggacaggcaaggaatacaggtattttg

SEQ ID NO: 49 - DNA, 34 bp, Artificial Sequence
  Factor V reverse site directed mutagenesis PCR primer
caaaatacctgtattccttgcctgtccagggatc

SEQ ID NO: 50 - DNA, 30 bp, Artificial Sequence; identical to SEQ ID NO: 45
  CCR2 matched oligonucleotide for Tm analysis

SEQ ID NO: 51 - DNA, 30 bp, Artificial Sequence
  CCR2 mismatched oligonucleotide for Tm analysis
ttattaagatgaggacgaccagcatgttgc

SEQ ID NO: 52 - DNA, 27 bp, Artificial Sequence
  CCR2 matched oligonucleotide for Tm analysis
caacatgctggtcgtcctcatcttaat

SEQ ID NO: 53 - DNA, 30 bp, Artificial Sequence; identical to SEQ ID NO: 44
  CCR2 mismatched oligonucleotide for Tm analysis

SEQ ID NO: 54 - DNA, 27 bp, Artificial Sequence
  CCR2 matched oligonucleotide for Tm analysis
caacatgctggtcgtcctcatcttaat

SEQ ID NO: 55 - DNA, 30 bp, Artificial Sequence; identical to SEQ ID NO: 44
  CCR2 mismatched oligonucleotide for Tm analysis

SEQ ID NO: 56 - DNA, 33 bp, Artificial Sequence
  CCR5 matched oligonucleotide for Tm analysis
gatgactatctttaatgtatggaaaatgagagc

SEQ ID NO: 57 - DNA, 31 bp, Artificial Sequence
  CCR5 mismatched oligonucleotide for Tm analysis
gactatctttaatgtctggaaattcttccag

SEQ ID NO: 58 - DNA, 33 bp, Artificial Sequence
  CCR5 matched oligonucleotide for Tm analysis
tctggaaattcttccagaattgatactgactgt

SEQ ID NO: 59 - DNA, 33 bp, Artificial Sequence; identical to SEQ ID NO: 56
  CCR5 mismatched oligonucleotide for Tm analysis

SEQ ID NO: 60 - DNA, 27 bp, Artificial Sequence
  SDF1 matched oligonucleotide for Tm analysis
tcccagaagaggcagacctggctccca

SEQ ID NO: 61 - DNA, 28 bp, Artificial Sequence
  SDF1 mismatched oligonucleotide for Tm analysis
ctcccagaagaggcagacccggctccca

SEQ ID NO: 62 - DNA, 25 bp, Artificial Sequence
  SDF1 matched oligonucleotide for Tm analysis
cacatgggagccgggtctgcctctt

SEQ ID NO: 63 - DNA, 30 bp, Artificial Sequence; identical to SEQ ID NO: 46
  SDF1 mismatched oligonucleotide for Tm analysis

SEQ ID NO: 64 - DNA, 26 bp, Artificial Sequence
  Factor V matched oligonucleotide for Tm analysis
cctctgtattccttgcctgtccaggg

SEQ ID NO: 65 - DNA, 27 bp, Artificial Sequence
  Factor V mismatched oligonucleotide for Tm analysis
tacctgtattcctcgcctgtccaggga

SEQ ID NO: 66 - DNA, 25 bp, Artificial Sequence
  Factor V matched oligonucleotide for Tm analysis
ccctggacaggcgaggaatacaggt

SEQ ID NO: 67 - DNA, 26 bp, Artificial Sequence
  Factor V mismatched oligonucleotide for Tm analysis
ccctggacaggcaaggaatacagagg

SEQ ID NO: 68 - DNA, 31 bp, Artificial Sequence
  MTHFR matched oligonucleotide for Tm analysis
ggtgtctgcgggagtcgatttcatcatcacg

SEQ ID NO: 69 - DNA, 31 bp, Artificial Sequence
  MTHFR mismatched oligonucleotide for Tm analysis
ggtgtctgcgggagccgatttcatcatcacg

SEQ ID NO: 70 - DNA, 31 bp, Artificial Sequence
  MTHFR matched oligonucleotide for Tm analysis
cgtgatgatgaaatcggctcccgcagacacc

SEQ ID NO: 71 - DNA, 31 bp, Artificial Sequence
  MTHFR mismatched oligonucleotide for Tm analysis
cgtgatgatgaaatcgactcccgcagacacc

SEQ ID NO: 72 - DNA, 26 bp, Artificial Sequence
  Factor XIII matched oligonucleotide for Tm analysis
gacagcaccaagccctgaagctacat

SEQ ID NO: 73 - DNA, 26 bp, Artificial Sequence
  Factor XIII mismatched oligonucleotide for Tm analysis
gacagcaccacgccctgaagctacat

SEQ ID NO: 74 - DNA, 24 bp, Artificial Sequence
  Factor XIII matched oligonucleotide for Tm analysis
ctgcgcttcagggcgtggtgatca

SEQ ID NO: 75 - DNA, 24 bp, Artificial Sequence
  Factor XIII mismatched oligonucleotide for Tm analysis
ctgcgcttcagggcttggtgatca

(19) United States
(12) Patent Application Publication
(10) Pub. No.: US 2002/0037507 A1
(43) Pub. Date: Mar. 28, 2002

(54) Compositions, methods and kits for allele discrimination

(76) Inventors: Cindy R. WalkerPeach, Austin, TX (US); Xiuyuan Hu, San Diego, CA (US)

Correspondence Address: KATHLEEN MADDEN WILLIAMS, Palmer & Dodge, LLP, One Beacon Street, Boston, MA, 02108, US

(21) Appl. No.: 09/736,863

(22) Filed: Dec. 14, 2000

Related U.S. Application Data
(60) Provisional application No. 60/171,126, filed on Dec. 16, 1999.

(51) Int. Cl. C12Q 1/68; C12P 19/34; C07H 21/04

(52) U.S. Cl. 435/6; 435/91.1; 536/24.3

(57) ABSTRACT

The invention relates to sequence-specific polynucleotide probes, pairs of probes, the design of pairs of probes in relation to the strands of target nucleic acid, and coordinate sequence-specific pairs of polynucleotide primers, and also relates to kits containing such probes, probe pairs, primers, and primer pairs. The invention thus relates to kits and methods employing such polynucleotides. The invention relates to six specific allele discrimination kits. The specific kits are developed for the detection of four single base substitutions in the human CCR2, SDF1, Factor V, MTHFR, Factor XIII genes, and a 32-bp deletion in the human CCR5 gene. The capability of using two allele-specific molecular beacons in the same PCR solution enables the simultaneous determination of three possible allelic representations of a given sequence change in target DNA (Allele 1/Allele 1, Allele 2/Allele 2, Allele 1/Allele 2). It also definitively differentiates a true negative signal that is due to the absence of an allele from a false negative signal that results from PCR failure.

### FIELD OF INVENTION

The present invention relates to the detection of single nucleotide sequences changes in DNA.

### BACKGROUND

Single nucleotide sequence changes (substitution, deletion or insertion) are the largest source of human DNA diversity, with an estimated frequency of 1 in 1,000 base pairs (1). Many of the sequence changes have been identified as the cause of monogenic disorders or to be associated with genetic predisposition to multifactorial diseases including cancer, diabetes and cardiovascular diseases. The sequence changes also constitute the genetic basis for many non-disease traits such as obesity and an individual's response to drugs. Moreover, single nucleotide polymorphisms (SNPs) are valuable genetic markers for gene discovery, population studies and individual identification. The demand is growing in both the research and clinical diagnostic fields for high-throughput mutation detection methodologies that are sensitive enough to distinguish nucleic acid sequences differing by as little as a single nucleotide.

It is a goal in this art to detect various nucleic acid sequences in a biological sample, in which the sequences, as so-called target sequences, are present in small amounts relative to its existence amongst a wide variety of other nucleic acid species including RNA, DNA or both.

### SUMMARY OF THE INVENTION

The invention encompasses a purified polynucleotide selected from the group consisting of SEQ ID NOS. 1-31, and also encompasses a pair of purified polynucleotides for allele discrimination, polynucleotides selected from the group consisting of SEQ ID NO. 1 and SEQ ID NO. 2; SEQ ID NO. 1 and SEQ ID NO. 4; SEQ ID NO. 3 and SEQ ID NO. 2; SEQ ID NO. 3 and SEQ ID NO. 4, SEQ ID NO. 5 and SEQ ID NO.6; SEQ ID NO. 7 and SEQ ID NO. 8; SEQ ID NO. 9 and SEQ ID NO. 10; SEQ ID NO. 11 and SEQ ID NO. 12, and SEQ ID NO. 30 and SEQ ID NO. 31.

Preferably, in a pair of polynucleotides for allele discrimination, each pair comprises first and second differentially labeled polynucleotides.

Preferably, in a pair of polynucleotides for allele discrimination, each of the first and second differentially labeled polynucleotides comprises a pair of fluorophore/quencher labels such that the first polynucleotide comprises a first pair of fluorophore/quencher labels and the second polynucleotide comprises a second pair of fluorophore/quencher labels.

The invention also encompasses a pair of polynucleotide primers for a polymerase chain reaction (PCR) selected from the group consisting of SEQ ID NO. 13 and SEQ ID NO. 14; SEQ ID NO. 15 and SEQ ID NO. 16; SEQ ID NO. 17 and SEQ ID NO. 18; SEQ ID NO. 19 and SEQ ID NO. 20; SEQ ID NO. 19 and SEQ ID NO. 21; SEQ ID NO. 22 and SEQ ID NO. 23; SEQ ID NO. 24 and SEQ ID NO. 25; SEQ ID NO. 26 and SEQ ID NO. 27; and SEQ ID NO. 28 and SEQ ID NO. 29. These primers may be used for, among other things, amplifying DNA and/or RNA isolated from a sample derived from an individual, such as a bodily material. The primers may be used to amplify a polynucleotide isolated from an individual, such that the polynucleotide may then be subject to various techniques for elucidation of the polynucleotide sequence. In this way, mutations in the polynucleotide sequence may be detected and used for diagnosis or prognosis.

The invention also encompasses a kit for allele discrimination comprising a pair of polynucleotides for allele discrimination, as described herein, and packaging materials therefor.

Preferably, such a kit may also include a coordinate pair of polynucleotide primers, as described herein, and a DNA polymerase. A pair of polynucleotide primers, such as PCR primers, useful in the invention, will include a forward and a reverse primer, the forward primer being complementary to a first strand of the target DNA and positioned upstream of (5′ to) a region in the target gene to be amplified, and the reverse primer will be complementary to the second strand (or opposite strand of the first strand) and positioned downstream of (3′ to) a region to be amplified.

A “coordinate” pair of polynucleotides means that the pair is complementary to the same target gene or gene region, although not the identical sequence in that gene, as another pair (for example, a pair of probes is complementary to the same target gene as a pair of primers.)

The invention also encompasses a kit for performing a polymerase chain reaction comprising a pair of polynucleotide primers disclosed herein, a DNA polymerase, and packaging materials therefor.

Preferably, the kit may include a DNA polymerase that is thermostable, and may in addition, also include a buffer suitable for allele discrimination and polymerase chain reaction.

Kits described herein also may include three genotype-specific control templates: (1) allele 1 control that contains DNA of the first allele (allele 1) of a specific gene target, (2) allele 2 control that contains DNA of the second allele (allele 2) of a specific gene target, (3) mixed allele 1 and allele 2 control that contains DNA of both the allele 1 and allele 2 specific gene target. The control templates may be genomic DNA or cloned DNA fragments which may also include a DNA standard, which may be genomic DNA, such as mouse genomic DNA.

A kit useful according to the invention for allele discrimination may include a pair of polynucleotides for allele discrimination of the CCR2-64I mutation, a G to A substitution at nucleotide position 190 (counting from the ATG start codon) of the CCR2 gene that results in a valine to isoleucine substitution at amino acid position 64 in the CCR2 gene, wherein a first polynucleotide of the pair has the sequence presented in SEQ ID NOS: 1 and a second polynucleotide of the pair of polynucleotides has the sequence presented in SEQ ID NOS: 2 or 4, a pair of polynucleotides for PCR of a region of the CCR2 gene wherein a first polynucleotide of the pair has the sequence presented in SEQ ID NO: 13 and a second polynucleotide of the pair of polynucleotides has the sequence presented in SEQ ID NO: 14, a DNA polymerase, and a buffer suitable for allele discrimination and polymerase chain reaction.

A kit useful according to the invention for allele discrimination may include a pair of polynucleotides for allele discrimination of the CCR2-64I mutation, a G to A substitution at nucleotide position 190 (counting from the ATG start codon) of the CCR2 gene that results in a valine to isoleucine substitution at amino acid position 64 in the CCR2 gene, wherein a first polynucleotide of the pair has the sequence presented in SEQ ID NOS: 3 and a second polynucleotide of the pair of polynucleotides has the sequence presented in SEQ ID NOS: 2 or 4, a pair of polynucleotides for PCR of a region of the CCR2 gene wherein a first polynucleotide of the pair has the sequence presented in SEQ ID NO: 13 and a second polynucleotide of the pair of polynucleotides has the sequence presented in SEQ ID NO: 14, a DNA polymerase, and a buffer suitable for allele discrimination and polymerase chain reaction.

Another kit useful according to the invention for allele discrimination may include a pair of polynucleotides for allele discrimination of the CCR5-del32 mutation, a 32 base pair deletion in the coding region of the CCR5 gene, wherein a first polynucleotide of the pair has the sequence presented in SEQ ID NO: 5 and a second polynucleotide of said pair of polynucleotides has the sequence presented in SEQ ID NO: 6, a pair of polynucleotides for PCR of a region of the CCR5 gene wherein a first polynucleotide of the pair has the sequence presented in SEQ ID NO: 15 and a second polynucleotide of the pair of polynucleotides has the sequence presented in SEQ ID NO: 16, a DNA polymerase, and a buffer suitable for allele discrimination and polymerase chain reaction.

Another kit useful according to the invention for allele discrimination may include a pair of polynucleotides for allele discrimination of the CCR5-del32 mutation, a 32 base pair deletion in the coding region of the CCR5 gene, wherein a first polynucleotide of the pair has the sequence presented in SEQ ID NO: 5 and a second polynucleotide of said pair of polynucleotides has the sequence presented in SEQ ID NO: 6, a pair of polynucleotides for PCR of a region of the CCR5 gene wherein a first polynucleotide of the pair has the sequence presented in SEQ ID NO: 17 and a second polynucleotide of the pair of polynucleotides has the sequence presented in SEQ ID NO: 18, a DNA polymerase, and a buffer suitable for allele discrimination and polymerase chain reaction.

Another kit useful according to the invention for allele discrimination may include a pair of polynucleotides for allele discrimination of a G to A substitution in the 3′ untranslated region (SDF1-3′A) of the SDF1 gene, wherein a first polynucleotide of the pair has the sequence presented in SEQ ID NO: 7 and a second polynucleotide of said pair of polynucleotides has the sequence presented in SEQ ID NO: 8, a pair of polynucleotides for PCR of a region of the SDF1 gene wherein a first polynucleotide of the pair has the sequence presented in SEQ ID NO: 19 and a second polynucleotide of the pair of polynucleotides has the sequence presented in SEQ ID NOS: 20, a DNA polymerase, and a buffer suitable for allele discrimination and polymerase chain reaction.

Another kit useful according to the invention for allele discrimination may include a pair of polynucleotides for allele discrimination of a G to A substitution in the 3′ untranslated region (SDF1-3′A) of the SDF1 gene, wherein a first polynucleotide of the pair has the sequence presented in SEQ ID NO: 7 and a second polynucleotide of said pair of polynucleotides has the sequence presented in SEQ ID NO: 8, a pair of polynucleotides for PCR of a region of the SDF1 gene wherein a first polynucleotide of the pair has the sequence presented in SEQ ID NO: 19 and a second polynucleotide of the pair of polynucleotides has the sequence presented in SEQ ID NOS: 21, a DNA polymerase, and a buffer suitable for allele discrimination and polymerase chain reaction.

Another kit useful according to the invention for allele discrimination may include a pair of polynucleotides for allele discrimination of the Factor V Leiden mutation, a G to A substitution of the Factor V gene that results in an arginine to glutamine change at amino acid position 506, wherein a first polynucleotide of the pair has the sequence presented in SEQ ID NO: 9 and a second polynucleotide of the pair of polynucleotides has the sequence presented in SEQ ID NO: 10, a pair of polynucleotides for PCR of a region of the Factor V gene wherein a first polynucleotide of the pair has the sequence presented in SEQ ID NO: 22 and a second polynucleotide of the pair of polynucleotides has the sequence presented in SEQ ID NO: 23, a DNA polymerase, and a buffer suitable for allele discrimination and polymerase chain reaction.

Another kit useful according to the invention for allele discrimination may include a pair of polynucleotides for allele discrimination of a C to T substitution at nucleotide position 677 of the human methylenetetrahydrofolate reductase (MTHFR) gene that results in an alanine to valine change, wherein a first polynucleotide of the pair has the sequence presented in SEQ ID NO: 11 and a second polynucleotide of the pair of polynucleotides has the sequence presented in SEQ ID NO: 12, a pair of polynucleotides for PCR of a region of the MTHFR gene wherein a first polynucleotide of the pair has the sequence presented in SEQ ID NO: 24 and a second polynucleotide of the pair of polynucleotides has the sequence presented in SEQ ID NO: 25, a DNA polymerase, and a buffer suitable for allele discrimination and polymerase chain reaction.

Another kit useful according to the invention for allele discrimination may include a pair of polynucleotides for allele discrimination of a C to T substitution at nucleotide position 677 of the human methylenetetrahydrofolate reductase (MTHFR) gene that results in an alanine to valine change, wherein a first polynucleotide of the pair has the sequence presented in SEQ ID NO: 11 and a second polynucleotide of the pair of polynucleotides has the sequence presented in SEQ ID NO: 12, a pair of polynucleotides for PCR of a region of the MTHFR gene wherein a first polynucleotide of the pair has the sequence presented in SEQ ID NO: 26 and a second polynucleotide of the pair of polynucleotides has the sequence presented in SEQ ID NO: 27, a DNA polymerase, and a buffer suitable for allele discrimination and polymerase chain reaction.

Another kit useful according to the invention for allele discrimination may include a pair of polynucleotides for allele discrimination of a G to T substitution at nucleotide 103 of the Factor XIII gene that results in a valine to leucine change, wherein a first polynucleotide of the pair has the sequence presented in SEQ ID NO: 30 and a second polynucleotide of the pair of polynucleotides has the sequence presented in SEQ ID NO: 31, a pair of polynucleotides for PCR of a region of the Factor XIII gene wherein a first polynucleotide of the pair has the sequence presented in SEQ ID NO: 28 and a second polynucleotide of the pair of polynucleotides has the sequence presented in SEQ ID NO: 29, a DNA polymerase, and a buffer suitable for allele discrimination and polymerase chain reaction.

Preferably, in any of the above kits, three genotype-specific control templates may be present: (1) allele 1 control that contains DNA of the first allele (allele 1) of a specific gene target, (2) allele 2 control that contains DNA of the second allele (allele 2) of a specific gene target, (3) mixed allele 1 and allele 2 control that contains DNA of both the allele 1 and allele 2 specific gene target. The control templates may be genomic DNA or cloned DNA fragments which may also include a DNA standard, which may be genomic DNA, such as mouse genomic DNA.

The invention also encompasses a method for allele discrimination, comprising the steps of: a) contacting a target nucleic acid with a pair of polynucleotides for allele discrimination as described herein, wherein the target nucleic acid comprises a sequence complementary to at least one polynucleotide of the pair, under conditions which permit formation of a hybrid between the target nucleic acid and at least one polynucleotide of the pair; and b) detecting the hybrid.

In this method, the pair of polynucleotides may be differentially labeled, and the first and second polynucleotides of the pair of polynucleotides may be differentially fluorescently labeled.

Thus, in this method, the detecting step may include detecting emission or quenching of fluorescence.

The invention also encompasses a method for amplifying a target nucleic acid, comprising the steps of: a) contacting a target nucleic acid with a pair of polynucleotide primers as described herein, wherein the pair of primers comprises forward and reverse primers for initiating a polymerase chain reaction, under conditions which permit formation of a hybrid between the pair of polynucleotide primers and the target nucleic acid; and b) extending the pair of polynucleotide primers in a polymerase chain reaction to form a PCR nucleic acid product that is complementary to the target nucleic acid.

The invention also encompasses a method for allele discrimination, comprising the steps of: a) contacting a target nucleic acid with a pair of polynucleotides for allele discrimination as described herein and a coordinate pair of polynucleotide primers for PCR, wherein the pair of primers comprises forward and reverse primers for initiating a polymerase chain reaction on the target DNA, wherein the target nucleic acid comprises a sequence complementary to at least one polynucleotide of the pair of polynucleotides for allele discrimination, under conditions which permit formation of a hybrid between the target nucleic acid and at least one polynucleotide of the pair for allele discrimination, and conditions also permitting formation of a hybrid between the target nucleic acid and the pair of polynucleotide primers; b) incubating the mixture of step (a) under conditions which permit a polymerase chain reaction to generate a PCR product that is complementary to the target nucleic acid and generation of a signal upon formation of a hybrid between the at least one polynucleotide of the pair for allele discrimination and the PCR nucleic acid product; and c) detecting the signal thereof.

The invention also encompasses a pair of polynucleotide probes wherein a first probe of the pair is complementary to the positive strand of a DNA duplex and a second probe of the pair is complementary to the negative strand of said DNA duplex.

Preferably, the loops of the pair of probes are non-complementary over 1 or more contiguous nucleotides. Furthermore, the stems of the pair of probes are preferably non-complementary.

Compositions, kits and methods described have advantages over many existing mutation detection methodologies. First, the use of hairpin shaped molecular beacons having the sequences specified herein are more sensitive as hybridization probes than the linear probes for detecting single nucleotide sequence changes. Second, because the kits and methods may be performed in a closed tube and no post-PCR manipulation of samples is required, the risk of PCR product carry-over contamination is greatly reduced. Furthermore, the time and effort involved in the test are significantly reduced. Third, the capability of using two allele-specific molecular beacons in the same PCR solution enables the simultaneous determination of three possible allelic representations of a two sequence variants in target DNA (Allele 1/Allele 1, Allele 2/Allele 2, Allele 1/Allele 2). It also definitively discriminates a true negative result from a false negative result that is due to PCR failure. Fourth, the design of two allele-specific beacons such that the loops of the beacons complement opposite strands of the target DNA offers better discrimination of mismatched probe/target hybrids than probes that complement the same strand. For example, this design enables probes to be targeted against a less stable C/A mismatch rather than a more stable G/T mismatch, thereby providing more discriminative probes.

Further features and advantages of the invention are described below.

### BRIEF DESCRIPTION OF THE DRAWINGS

FIG. 1A schematically illustrates detection of single nucleotide sequence change by molecular beacon. The wild type and mutant DNA differ from each other by a single base pair. The hairpin shaped molecular beacon consists of a probe sequence that perfectly matches with the mutant DNA but mismatches with the wild type DNA by a single nucleotide (A to C). The beacon cannot form a stable probe/target hybrid with the wild type DNA at given temperature and thus adopts the closed structure. As a result, the fluorescence of the fluorophore is quenched. In contrast, the beacon can form a stable hybrid with its perfectly matched mutant DNA and thus the fluorescence of the fluorophore is restored.

FIG. 1B shows simultaneous determination of three allelic representations of a given sequence variation. Two allele-specific molecular beacons (green color for wild type and orange color for mutant) are added to the same PCR solution. The detection of only the green fluorescence signal after PCR indicates the presence of only the wild type allele (W/W) in the target DNA. The detection of only the orange fluorescence signal indicates the presence of only the mutant allele (M/M). The detection of both fluorescence signals indicates the presence of both alleles (W/M).

FIG. 2A Schematic representation of molecular beacons designed to detect an A-to-C single nucleotide polymorphism using probes directed to the opposite strand.

FIG. 2B Schematic representation of molecular beacons designed to detect an A-to-C single nucleotide polymorphism using probes directed to the same strand.

### DESCRIPTION

The invention is based on compositions, kits and methods useful for allele discrimination; i.e., to detect human gene mutations. The invention therefore provides polynucleotide pairs, in which the members of a pair differ at a single nucleotide; in labeled form, these polynucleotide pairs are called allele-specific molecular beacons. The invention also provides a pair of polynucleotides which serve as primers for extending and completing the nucleic acid which lies between the primers, so as to generate a PCR product.

### Definitions

A “polynucleotide probe”, as used herein, means a polynucleotide of any length, but preferably about 15-60, preferably 20-40 nucleotides in length, which is capable of hybridizing to and thus detecting a nucleic acid target sequence, or a target gene. The probe contains a sequence of about 10-35 nucleotides surrounded by arm sequences of about 4-8 nucleotides which are complementary to each other, wherein the formation of a self-hybrid (a duplex between the arm sequences) or alternatively the failure to form a self-hybrid in the arms produces a detectable signal if the probe is hybridized to a target. The term “molecular beacon” encompasses a probe that is labeled with a detectable label, the label typically being a fluorescent label on one end of the polynucleotide and a quencher (fluorescent or non-fluorescent) on the other end of the polynucleotide, such that quenching of the label occurs when the arm sequences are self-hybridized, and fluorescence emission occurs when the arm sequences are not self-hybridized, but rather the probe is hybridized to a target sequence.

As used herein, “stem” refers to the self-hybrid duplex formed between the arm sequences of polynucleotide probes.

As used herein, “loop” refers to the sequence of 10-35 nucleotides of a polynucleotide probe that is surrounded by arm sequences, such that the loop is a single-stranded portion of the polynucleotide probe that is fully complementary to the region of the target nucleic acid to which the polynucleotide probe binds.

“Complementary” refers to the ability of a nucleic acid single strand (or portion thereof) to hybridize to an anti-parallel nucleic acid single strand (or portion thereof) by contiguous base-pairing between the nucleotides (that is not interrupted by any unpaired nucleotides) of the anti-parallel nucleic acid single strands, thereby forming double stranded nucleic acid between the complementary strands.

The terms “first” and “second” strand refer to the strands of a double-stranded nucleic acid, where one strand can be regarded as the first strand, and its complementary strand can be regarded as the second strand. Alternatively, the two nucleic acid strands of the double-stranded nucleic acid may be referred to as the 5′ to 3′ strand and its complement, the 3′ to 5′ strand.

As used herein, “positive” or “sense” strand refers to the strand of the DNA duplex of a gene that contains the sequence of the corresponding mRNA transcript of the gene.

“Negative strand” or “antisense” strand refers to the strand of the DNA duplex of a gene that contains the sequence that is complementary to the corresponding mRNA transcript of the gene.

A “target” nucleic acid or sample refers to the nucleic acid used for analysis and to which a polynucleotide for allele discrimination and/or a pair of PCR primers is hybridized in order to ascertain the presence or absence of a mutation or polymorphism in the target nucleic acid.

As used herein, “forward amplification primer” refers to an oligonucleotide used for PCR amplification that is complementary to the sense strand of the target nucleic acid. “Reverse amplification primer” refers to an oligonucleotide used for PCR amplification that is complementary to the antisense strand of the target nucleic acid. For a given target, a forward and reverse amplification primer are used to amplify the DNA in PCR.

A “sample” refers to a target nucleic acid, and may consists of purified or isolated nucleic acid, or may comprise a biological sample containing nucleic acid, such as a tissue sample, a biological fluid sample, a cell sample.

“Control” DNA refers to both total human genomic DNA and a cloned human genomic DNA fragment that encompasses the region of the target nucleic acid DNA in a control reaction. Control DNA is ideally genomic DNA if the sample to be tested is genomic DNA; alternatively the control is a cloned genomic DNA fragment preferably in the format of a mixture of the cloned DNA and a “DNA standard” of genomic DNA, which may be mouse genomic DNA. The mixture of the control cloned DNA and a DNA standard provides sufficient complexity to mimic the complexity of the genomic DNA sample to be tested. Alternatively, if the sample to be tested is plasmid DNA or mitochondrial DNA, then control DNA may be plasmid or mitochondrial DNA, respectively.

“Template” DNA refers to a recombinant DNA which encompasses the region of the target sequence that the probes and primers are complementary to.

As used herein, a “control DNA template” refers to the sequence-matched or mismatched targets useful in the invention.

The term “isolated”, when used in reference to a nucleic acid means that a naturally occurring sequence has been removed from its normal cellular (e.g., chromosomal) environment or is synthesized in a non-natural environment (e.g., artificially synthesized). Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

As used herein, the term “polynucleotide(s)” generally refers to any polyribonucleotide or poly-deoxyribonucleotide, which may be unmodified RNA or DNA or modified RNA or DNA. “Polynucleotide(s)” include, without limitation, single- and double-stranded nucleic acids. As used herein, the term “polynucleotide(s)”also includes DNAs or RNAs as described above that contain one or more modified bases. Thus, DNAs or RNAs with backbones modified for stability or for other reasons are “polynucleotide(s)”. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

As used herein, “oligonucleotide primers” refer to single stranded DNA or RNA molecules that are hybridizable to a nucleic acid template and prime enzymatic synthesis of a second nucleic acid strand.

“Mutation” refers to a substitution, deletion, or insertion, and may involve a single nucleotide or two or more nucleotides in a given sequence.

“Single nucleotide polymorphism” (SNP) refers to a single nucleotide alteration of a wild type sequence, that may be a substitution, deletion, or insertion.

As used herein, “deletion” refers to a change in either a nucleotide or amino acid sequence wherein one or more nucleotides or amino acid residues, respectively, are absent.

As used herein, “insertion” or “addition” refers to a change in either nucleotide or amino acid sequence wherein one or more nucleotides or amino acid residues, respectively, have been added.

As used herein, “substitution” refers to a replacement of one or more nucleotides or amino acids by different nucleotides or amino acid residues, respectively.

As used herein, “comparing” a sequence refers to determining if the nucleotides at one or more positions in a particular region of a nucleic acid fragment are identical for any two or more sequences.

As used herein, “amplifying” refers to producing additional copies of a nucleic acid sequence, preferably by the method of polymerase chain reaction (Mullis and Faloona, 1987, Methods Enzymol., 155: 335).

“PCR product” refers to the nucleic acid generated from PCR amplification of a given region of a target nucleic acid.

A “gene” refers to a coding region of DNA, not including the regulatory regions upstream and downstream of the coding region. “Gene” refers to the genomic gene, including introns and exons, and also may refer to cDNA, including the exons only. “Regulatory region(s)” refers to the regions upstream and/or downstream of a coding region, such as a promoter and enhancer.

“Hybrid” or “complex” refers to a double-stranded nucleic acid, i.e., duplex DNA or RNA or DNA/RNA, which is fully complementary in the region of the nucleic acid which is double-stranded. The terms also are meant to include a duplex wherein a target nucleic acid is duplexed with a pair of forward and reverse primers, as well as with at least one polynucleotide for allele discrimination, such as a molecular beacon.

“Recombinant” refers to a nucleic acid (DNA or RNA) which has been genetically altered and/or recombined via genetic engineering, and not by natural genetic mutation and/or recombination.

### Kits

Kits according to the invention thus will include a pair of molecular beacons having the sequences disclosed herein and/or a coordinate pair of PCR primers having sequences disclosed herein. Optionally, a kit useful according to the invention also may contain one or more of a pair of allele-specific DNA targets (wild type and mutant or allele 1 and allele 2), dNTPs, a DNA polymerase, for example, the Taq2000 DNA polymerase and PCR buffer. The term “allele-specific DNA targets” refers to target DNA which contains both alleles of a sequence variation under investigation and therefore serve as allele-specific positive controls.

In the inventive methods, a nucleic acid sample to be analyzed is added to a kit, and a signal is generated if one or both molecular beacons hybridizes to its target sequence.

The sequence-specific kits disclosed herein are developed for the detection of six mutations in the human CCR2, CCR5, SDF1, Factor V, MTHFR and Factor XIII genes. These mutations are present at high frequencies in the population and have important medical and biological implications.

(1) CCR2, CCR5 and SDF1 allele discrimination kits

The human CCR5 gene encodes a cell surface chemokine receptor molecule that serves as the principal co-receptor, with CD4, for macrophage-tropic strains of human immunodeficiency virus-type 1 (HIV-1) (33). A common 32-base pair deletion in the CCR5 gene that causes truncation and loss of CCR5 receptors on lymphoid cell surfaces of the mutant homozygotes has been described (9). Genetic analysis indicated that homozygotes for the mutation are highly resistant to HIV-1 infection and the heterozygotes may also delay the progression to AIDS in infected individuals (9, 11, 12). This mutation has been identified in different ethnic groups around the world with highest allele frequency (10-20%) recorded in European population (10).

The human CCR2 gene encodes a cell surface chemokine receptor that serves as a critical co-receptor for certain macrophage-tropic, T lymphocyte-tropic, and dual-tropic strains of HIV (7). A G-to-A nucleotide substitution was detected at position 190 (counting from the ATG start codon) that results in valine to isoleucine substitution at position 64 (7). Genetic analysis indicated that HIV-1 infected individuals carrying the mutation progressed to AIDS 2 to 4 years later than individuals homozygous for the common (wild-type) allele (7,8). This mutation occurred at an allele frequency of 10 to 15% among Caucasians and African Americans (7).

Stromal-derived factor (SDF-1) is the principal ligand for CXCR4, a co-receptor with CD4 for T lymphocyte-tropic strains of HIV-1. A G-to-A nucleotide substitution at position 801 (counting from the ATG start codon) in the 3′ untranslated region of the gene was identified in different ethnic groups (allele frequencies: 5-25%) (13). Genetic studies indicated that the mutant homozygotes can either delay (13, 34) or accelerate (14) the onset of AIDS in HIV-1 infected individuals. The SDF-1 variant (SDF1-3′A) is located in the 3′ untranslated region that is highly conserved in sequence between mouse and human. It has been suggested that this variant may be responsible for up-regulation of the quantity of the SDF-1 protein available to bind to CXCR4, the co-receptor for T-tropic strains of HIV (13).

A common variant in the promoter region of the CCR5 gene (CCR5P1) has recently been found to accelerate the onset of AIDS (34). The detection of the CCR2, CCR5, SDF-1 alleles that confer AIDS protection and the CCR5P1 allele that accelerates the AIDS onset is of significant research and diagnostic values for this disease.

(2) Factor V allele discrimination kit

The human coagulation factor V (factor V) gene encodes a protein that is an essential component of the blood coagulation cascade (15, 35). During coagulation, the factor V protein is converted to the active cofactor, factor Va. During normal haemostasis, activated protein C (APC), a serine protease with potent anti-coagulant properties, limits clot formation by proteolytic inactivation of factor Va and VIIIa. More than half of all patients with familial or recurring venous thrombosis was found to have hereditary resistance to APC. The resistance was later found to result from a point mutation occurred in the factor V gene (factor V Leiden) (15). This mutation, a G-to-A substitution at nucleotide position 1,691, converts amino acid 506 from an arginine to a glutamine. Because the mutant factor Va protein cannot be cleaved and inactivated by APC, carriers of this mutation are at significantly increased risk of venous thrombosis. Hereditary resistance to APC is by far the most common genetic risk factor for venous thrombosis. The thrombotic complications of inappropriate coagulation are very common, affecting ˜300,000 Americans annually (16). The allele frequency of this mutation was found to be 2˜5% in Caucasian population (15, 36). The factor V molecular beacon allele discrimination kit is also useful for high volume screening of this mutation.

(3) MTHFR allele discrimination kit

Methylenetetrahydrofolate reductase (MTHFR) catalyses the reduction of methylenetetrahydrofolate to methyltetrahydrofolate, a cofactor for methylation of homocysteine to methionine. Deficiency of MTHFR results in hyperhomocycteinemia, which has been identified as a risk factor for cerebrovascular, peripheral vascular and coronary heart disease (17-19). A common mutation, a C to T substitution at nucleotide position 677, has been identified in the MTHFR gene (17). This mutation converts an alanine to valine residue. The mutation in the heterozygous or homozygous state correlates with reduced enzyme activity and individuals homozygous for the mutation have significantly elevated plasma homocysteine levels. Thus, this mutation may represent an important genetic risk factor in vascular disease. The allele frequency of this mutation was found to be 38% in Caucasians (17). The MTHFR allele discrimination kit permits the detection of this mutation.

(4) Factor XIII allele discrimination kit

The human coagulation factor XIII (factor XIII) gene encodes a protein that is an essential component of the blood coagulation cascade. Human Factor XIII, the fibrin-stabilizing factor, is a plasma transglutaminase that catalyzes fibrin-fibrin crosslinking. Additionally, the enzyme is responsible for crosslinking fibrin to other proteins such as fibronectin, α2-plasmin inhibitor, and collagen. FXIII circulates in blood as a heterotetramer proenzyme consisting of two catalytic A (FXIIIA) and two noncatalytic B (FXIIIB) subunits. During the final stages of coagulation, the proenzyme is activated by thrombin cleavage (37, 38, 39). While there are more than 20 described polymorphisms in the FXIIIA gene, the only one to have shown a predictive risk value is a G-to-T mutation that converts amino acid 34 from a valine to a leucine (40). This mutation has been found associated with a decreased incidence of myocardial infarcation (38, 39) and protection against venous thrombosis (41), but predisposes to intracranial hemorrhage (40). In Caucasian populations, the T-allele frequency is 21-28% (38, 39).

TABLE 1
Gene Targets for Molecular Beacon Allele Discrimination Kits

| | Protein | | Allele | Consequence |
|---|---|---|---|---|
| Gene | Function | Mutation | Frequency | of Mutation |
| CCR2 | Chemokine | G to A | Caucasians | Delays AIDS |
| | receptor and a | substitution | (˜10%)⁷ | onset in |
| | co-receptor for | that results | African | HIV-1 |
| | certain M-, T-, | in Val to Ile | Americans | infected |
| | and dual-tropic | change at | (˜15%)⁷ | individuals^{7.8} |
| | strains of HIV. | position 64 | Hispanics | |
| | | (CCR2-64I)⁷ | (˜17%)⁷ | |
| | | | Asians | |
| | | | (˜25%)⁷ | |
| CCR5 | Chemokine | A 32-bp | Ashkenazi | Delays AIDS |
| | receptor and | deletion that | Jews | onset in |
| | major co- | results in a | (˜20%)¹⁰ | HIV-1 |
| | receptor for T- | truncated | Europeans | infected indi- |
| | tropic HIV-1 | protein⁹ | (2-15%)¹⁰ | viduals^{9,11,12} |
| | | | Asians | |
| | | | (0-10%)¹⁰ | |
| | | | Africans | |
| | | | (0-0.5%)¹⁰ | |
| SDF1 | Chemokine and | G to A | Caucasians | Mutant |
| (Stromal- | natural ligand | substitution | (˜20%)¹³ | homozygote |
| derived | of CXCR4, the | in the 3′ un- | Hispanics | (3′A/3′A) is |
| Factor-1) | major co- | translated | (˜16%)¹³ | associated |
| | receptor for T- | region | African | with |
| | tropic HIV-1 | (SDF1- | Americans | delayed¹³ or |
| | | 3′A)¹³ | (˜5%)¹³ | accelerated¹⁴ |
| | | | Asians | AIDS |
| | | | (˜25%)¹³ | progression. |
| Factor V | Inactivation | G to A | Caucasians | Mutant |
| | of activated | substitution | (2-5%)^{15,16} | carriers have |
| | protein C | that results | | increased |
| | (APC), which | in Arg to | | risk for |
| | is an anti- | Gln change | | venous |
| | coagulant | at position | | throm- |
| | enzyme | 506 (Factor | | bosis^{15,16} |
| | | V Leiden)¹⁵ | | |
| MTHFR | A key enzyme | C to T | Caucasians | Individuals |
| (Methylene- | in homo- | substitution | (38%)¹⁷ | of mutant |
| tetrahydro- | cysteine | at nucleotide | | homozygotes |
| folate | metabolism | position 677 | | have |
| reductase) | | that results | | elevated |
| | | in an Ala to | | plasma |
| | | Val change¹⁷ | | homo- |
| | | | | cysteine |
| | | | | level, a |
| | | | | major risk |
| | | | | factor for |
| | | | | vascular |
| | | | | diseases^{18,19} |
| Factor XIII | Activated form | G to C | Caucasians | Mutant |
| | cross links | substitution | (21- | carriers have |
| | fibrin | that results | 28%)^{38,39} | increased |
| | monomers in | in Val to Leu | | risk for |
| | coagulation | change at | | intracranial |
| | | amino acid | | hem- |
| | | position | | orrhage³⁷ |
| | | 34³⁷ | | |

### Preparation of Control DNA

The detection of nucleotide sequence changes by molecular beacons is based on the differential hybridization of the beacons with their sequence-matched or mismatched targets. Thus, both the sequence-matched and mismatched targets useful in the invention. In addition, these targets serve as “control DNA templates” in the inventive methods.

The control DNA templates were provided through PCR cloning and site-directed mutagenesis. Briefly, PCR was carried using human genomic DNA (Sigma) as the template to amplify a DNA fragment that spans the site of mutation. The PCR primer sequences were designed according to the DNA sequences in GENBANK. The PCR primers used and the sizes of the amplicons are listed in Table 2.

TABLE 2
Primers used for PCR cloning of DNA targets

| | | | |
|---|---|---|---|
| Gene | Primers | Amplicon size (bp) | Genbank Accession # |

| | | | |
|---|---|---|---|
| CCR2 | F: 5′-ATG CTG TCC ACA TCT CGT TC | 327 | U80924 |
| | R: 5′-CCC AAA GAC CCA CTC ATT TG | | |
| CCR5 | F: 5′-TGG CTG TGT TTG CGT CTC TC | 249 (Wild Type) | U54994 |
| | R: 5′-AGA TAA GCC TCA CAG CCC TG | 217 (Mutant) | |
| SDF1 | F: 5′-CAG TCA ACC TGG GCA AAG CC | 302 | L36033 |
| R: 5′-AGC TTT GGT CCT GAG AGT CC | | | |
| Factor V | F: 5′-TGC CCA GTG CTT AAC AAG ACC A | 267 | M16967 |
| | R: 5′-TGT TAT CAC ACT GGT GCT AA | | |
| MTHFR | F: 5′-CTT GAA CAG GTG GAG GCC AG | 301 | U09806 |
| | R: 5′-AGG ACG GTG CGG TGA GAG TG | | |
| Factor XIII | F: 5′-CCC AAT AAC TCT AAT GCA GCG | 91 | M21987 |
| | F: 5′-TGC TCA TAC CTT GCA GGT TG | | |

The TaqPlus™ Precision DNA polymerase (Stratagene) was selected for use in PCR due to its robust nature and high fidelity. Other DNA polymerases known in the art to be useful in PCR may also be used in the inventive kits and methods. The PCR amplicons were subsequently cloned into a plasmid vector (PCR-Blunt, Invitrogen) and the inserts were sequenced using the ThermoSequenase™ sequencing kit (Amersham Life Science). In four cases (CCR2, CCR5, SDF1 and factor V), the cloned DNA contained only the wild type allele and the mutant allele was generated experimentally for three of them (CCR2, SDF1, FV). To generate the mutant allele, site-directed mutagenesis was performed using the cloned wild type DNA as the template. The QuikChange™ site-directed mutagenesis kit (Stratagene) was used for making the sequence changes. The CCR5 mutant allele was generated by PCR cloning using a homozygous mutant DNA (Cenetron Diagnostics) as the template. In the case of MTHFR, the cloned DNA contained both the wild-type and the mutant alleles. The sequences for all of the wild-type and mutant alleles were confirmed by DNA sequence analysis. The primers used in the site-directed mutagenesis are listed in Table 3.

TABLE 3
Primers used for site-directed mutagenesis

| | |
|---|---|
| Gene | Primers |

| | |
|---|---|
| CCR2 | F: 5′-GGA ACA TGG TGG TCA TCC TCA TCT TAA TAA |
| | R: 5′-TTA TTA AGA TGA GGA TGA CCA GCA TGT TGC |
| SDF1 | F: 5′-TCC ACA TGG GAG CCA GGT GTG CCT CTT CTG |
| | R: 5′-GAG AAG AGG CAG ACC TGG CTC CCA TGT GGA |
| Factor V | F: 5′-GAT CCC TGG ACA GGC AAG GAA TAC AGG TAT TTT G |
| | R: 5′-CAA AAT ACC TGT ATT CCT TGC GTG TGG AGG GAT G |

Note: Letters in bold indicate the bases present in mutant allele

To provide control DNA of targets that would be amplified from genomic DNA, the control plasmid DNAs were mixed with mouse genomic DNA as a DNA standard to generate a genotype-specific DNA control for each kit. Mouse genomic DNA was purchased form Promega (Cat.#G3091). Three types of genotype-specific DNA controls were made: wild-type (WT), mutant (MT) and heterozygote (Het.). One μl of WT mouse standard contains approximately 10 ng of mouse genomic DNA and 1 to 5 pg of WT control plasmid. One μl of MT mouse standard contains approximately 10 ng of mouse genomic DNA and 1 to 10 pg of MT control plasmid. One μl of Het. mouse standard contains approximately 10 ng of mouse genomic DNA and 0.5 to 3 pg of each WT and MT control plasmid.

### Molecular Beacons

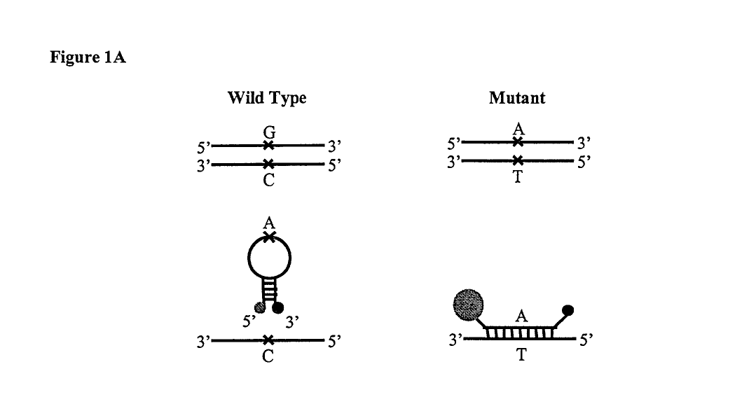

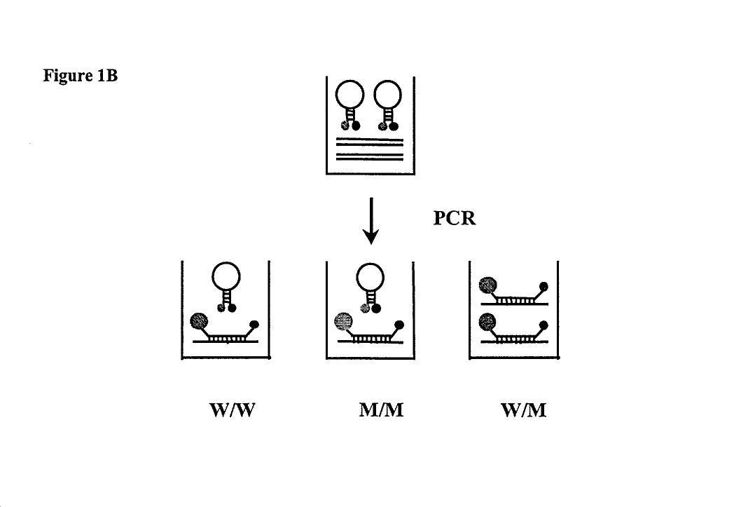

Molecular beacons are single-stranded oligonucleotide probes that possess a stem-and-loop hairpin structure. The loop portion of the molecule is a probe sequence complementary to a target sequence (e.g., an internal region of a PCR amplicon) and the stem is formed by short complementary sequences located at the opposite ends of the molecule. The molecule is labeled with a fluorophore at one end and a quencher at the other end. When free in solution, the stem keeps the fluorophore and the quencher in close proximity, causing the fluorescence of the fluorophore to be quenched by energy transfer. When bound to its complementary target the probe/target hybrid forces the stem to unwind, separating the fluorophore from the quencher, and restoring the fluorescence (FIG. 1A). A comparison of “hairpin probes” with sequence-matched “linear probes” demonstrates that the presence of the hairpin stem significantly enhances the specificity of molecular beacons, enabling them to distinguish targets that differ by as little as a single nucleotide (3). In addition, the hairpin conformation allows a variety of fluorophores to be used in conjunction with the same quencher. Thus, allele-specific molecular beacons, each labeled with a different fluorophore, can be used to detect several different target sequences present in the same solution. The capability of using two allele-specific molecular beacons in the same PCR solution enables the simultaneous determination of three possible allelic representations of a given sequence change in target DNA (FIG. 1B). It also definitively discriminates a true negative result from a false negative result that is due to PCR failure. Therefore, these probes are particularly suitable as hybridization probes for allele discrimination involving single base pair mismatches.

### Design of Molecular Beacons

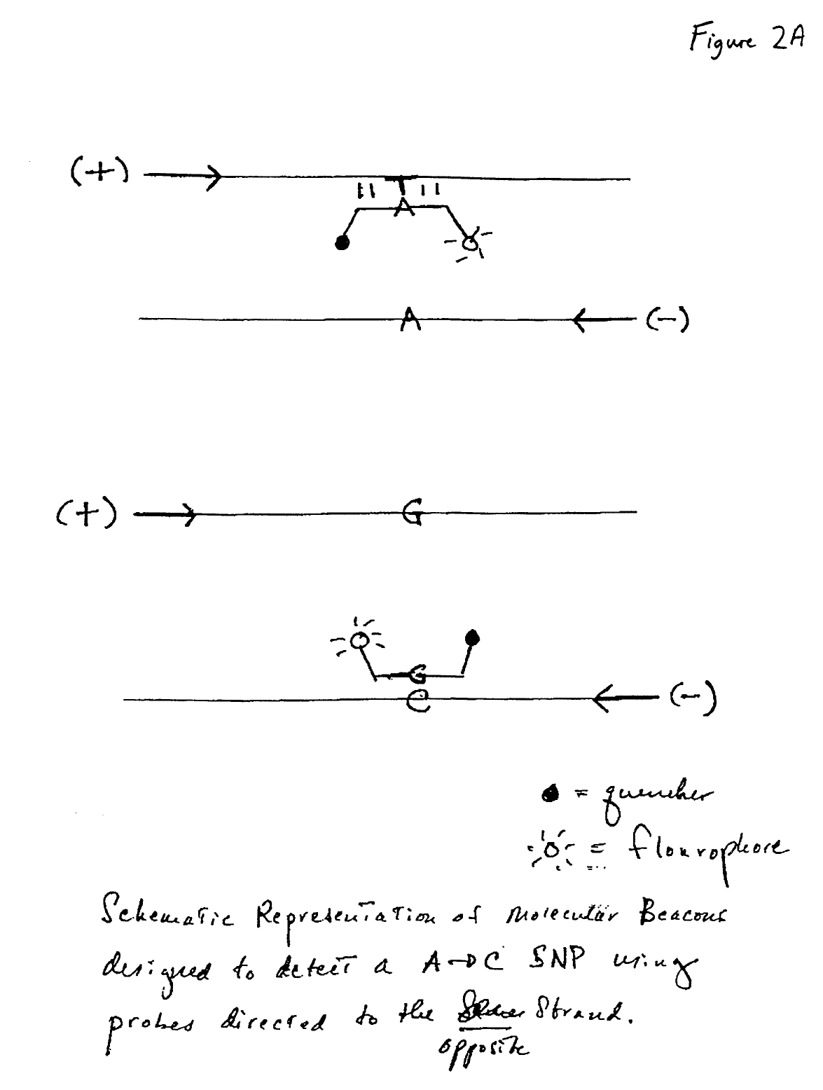

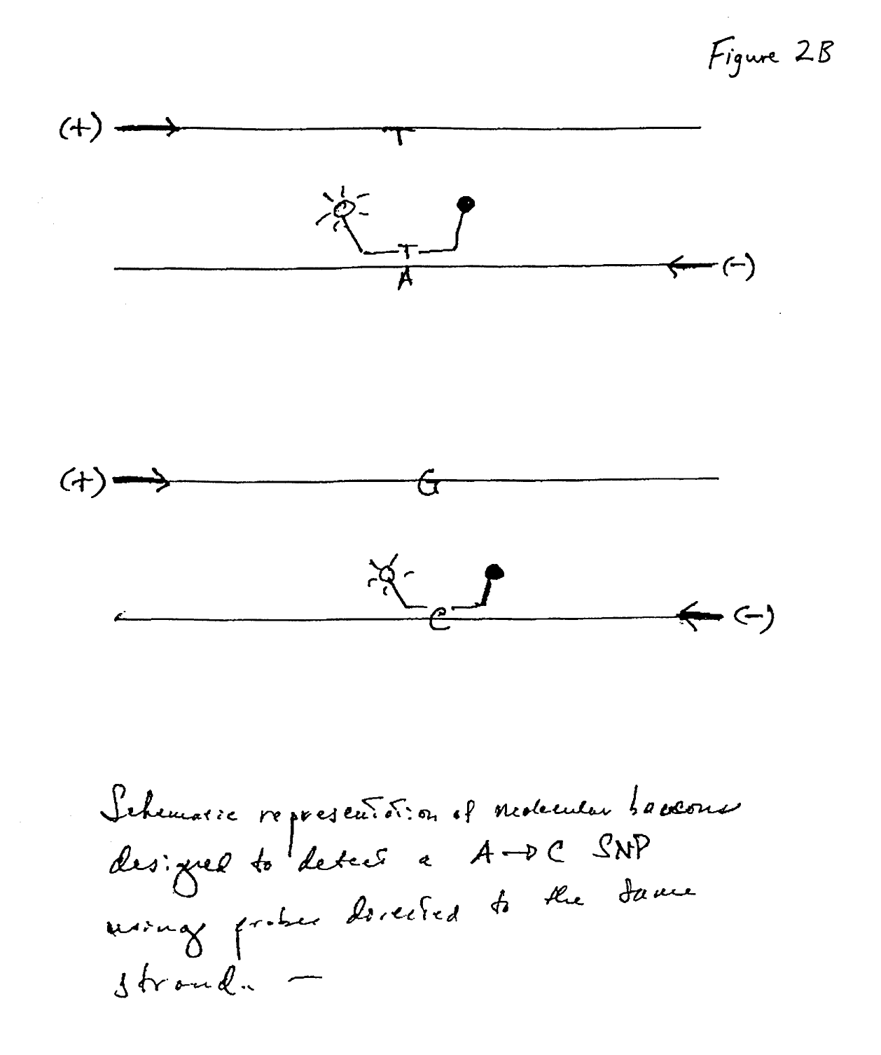

For the detection of a nucleotide sequence change, two molecular beacons with complete sequence match to either the wild type or the mutant sequence were generated. The two beacons were also designed to complement opposite strands of the target DNA, that is one complementary to the positive strand and one complementary to the negative strand (FIG. 2A) to provide better discrimination of mismatched probe/target hybrids than probes that complement the same strand (FIG. 2B). The two beacons were labeled with different fluorophores that emit fluorescent light at specific optical wavelength. As a result, the wild type and the mutant alleles that co-existed in the same PCR reaction can be distinguished. In most cases, the fluorophore TET (Tetrachloro-fluorescein) was used to label the wild type allele-specific beacons and FAM (6-carboxy--fluorescein) was used to label the mutant allele-specific beacons. These two fluorescent signals can be distinguished by using the ABI7700 sequence detector software. DABCYL was used as the quencher for all of the molecular beacons. Table 4 listed the sequences of the 14 molecular beacons used in the kits. These beacons were dissolved in TE buffer (10 mM Tris and 1 mM EDTA, pH 8.0) and stored in −20° C. freezer. The concentrations of all the beacons were determined by UV absorbance (260 nm) using a spectrophotometer (Beckman DU600).

TABLE 4
Sequences of Molecular Beacons

| | | | | | |
|---|---|---|---|---|---|
| | | | | | Strand |
| | | SEQ | | | Com- |
| | | ID | Fluoro | | ple- |
| Gene | Beacon Sequence | NO. | phore | Beacon Name | mented |

| | | | | | |
|---|---|---|---|---|---|
| CCR2 | M1: 5′GCG ACG CAT GCT GGT GAT CCT CAT CTT CGT CGC | 1 | FAM | Beacon-M/CCR2 | (−) |
| | W1: 5′CGC AGG ATG AGG ACG ACC AGC ACT GCG | 2 | TET | Beacon-W/CCR2a | (+) |
| | M2: 5′CGC ACC ATG CTG GTC ATC CTC ATG TGC G | 3 | FAM | Beacon-M/CCR2b | (−) |
| | W2: 5′CGC GTC TGA GGA CGA CCA GCA TGT TGG ACG CG | 4 | TET | Beacon-W/CCR2b | (+) |
| CCR5 | M: 5′GCG AGC TCA TTT TCC ATA CAT TAA AGA TAG TGC TCG C | 5 | FAM | Beacon-M/CCR5 | (−) |
| | W: 5′CGC ACG TCA GTA TCA ATT CTG GAA GAA TTT CCG TGC G | 6 | TET | Beacon-W/CCR5 | (−) |
| SDF1 | M: 5′CGC GTG CCA GGT CTG CCT CTT CTA CGC G | 7 | FAM | Beacon-M/SDF | (−) |
| | W: 5′CGA CGG ACC CGG CTC CCA TGC GTC G | 8 | TET | Beacon-W/SDF1 | (+) |
| Factor V | M: 5′CGA CGT GGA CAG GCA AGG AAT ACC GTC G | 9 | FAM | Beacon-M/FV | (−) |
| | W: 5′CGA CGT GTA TTC CTC GCC TGT CCG TCG | 10 | TET | Beacon-W/FV | (+) |
| MTHFR | M: 5′CCG CTT GAT GAA ATC GAC TCC CGA GCG G | 11 | FAM | Beacon-M/MTHFR | (+) |
| | W: 5′CCG GTG CGG GAG CCG ATT TCA ACC GG | 12 | TET | Beacon-W/MTHFR | (−) |
| Factor XIII | M: 5′CGC ACG CTT CAG GGC TTG GTG CCG TGC G | 30 | TET | Beacon-M/FXIII | (+) |
| | W: 5′GCG ACG CAC CAC GCC CTG AAG CCG TCG C | 31 | FAM | Beacon-W/FXIII | (−) |

(+) indicates positive or sense strand
(−) indicates negative or anti-sense strand

### Melting Curve Analysis

A melting curve analysis is carried out by incubating a molecular beacon with or without its sequence-matched or mismatched single stranded oligonucleotide target. In the analysis, three sets of duplicated sample mixture (50 μl) were prepared in the 0.2-ml PCR tubes (MicroAmp optical tubes, Perkin Elmer). The first set contains the beacon buffer (final concentration: 50 mM KCl, 4 mM MgCl₂ and 10 mM Tris, pH8.0), the molecular beacon (final concentration: 0.2 μM), and the single stranded oligonucleotide whose sequence completely matches that of the beacon (final concentrations: 0.4 μM/0.8 μM). The second set contains the same beacon buffer, the same molecular beacon (same concentration as set 1) and the single stranded oligonucleotide that consists of a mismatched nucleotide with the beacon (same concentration as set 1). The third set contains the beacon buffer and the beacon (same concentration as set land 2) but no oligonucleotide target. The beacon buffer was chosen as it mimics that used in PCR. Performed in the ABI7700 thermal cycler, the samples were first heated at 95° C. for 2 minutes followed by the change of incubation temperature from 85° C. to 20° C. at the speed of 1° C. per minute. The single stranded oligonucleotide targets used in the melting curve analysis are listed in Table 5.

TABLE 5
Oligonucleotides Used for Melting Curve Analysis

| | | |
|---|---|---|
| 1. | Beacon-M/CCR2: | 5′ GCG ACG CAT GCT GGT CAT CCT CAT CTT CGT CGC |
| | Matched oligo: | 5′ TTA TTA AGA TGA GGA TGA CCA GCA TGT TGC |
| | Mismatched oligo. | 5′ TTA TTA AGA TGA GGA CGA CCA GCA TGT TGC |
| 2. | Beacon-W/CCR2a: | 5′ CGC AGG ATG AGG ACG ACC AGC ACT GCG |
| | Matched oligo: | 5′ CAA CAT GCT GGT CGT CCT CAT CTT AAT |
| | Mismatched oligo: | 5′ GCA ACA TGC TGG TCA TCC TCA TCT TAA TAA |
| 3. | Beacon-W/CCR2b: | 5′ CGC GTC TGA GGA CGA CCA GCA TGT TGG ACG CG |
| | Matched oligo: | 5′ CAA CAT GCT GGT CGT CCT CAT CTT AAT |
| | Mismatched oligo: | 5′ GCA ACA TGC TGG TCA TCC TCA TCT TAA TAA |
| 4. | Beacon-M/CCR5: | 5′ GCG AGC TCA TTT TCC ATA CAT TAA AGA TAG TGC TCG C |
| | Matched oligo: | 5′ GAT GAC TAT CTT TAA TGT ATG GAA AAT GAG AGC |
| | Mismatched oligo: | 5′ GAC TAT CTT TAA TGT CTG GAA ATT CTT CCA G |
| 5. | Beacon-W/CCR5: | 5′ CGC ACG TCA GTA TCA ATT CTG GAA GAA TTT CCG TGC G |
| | Matched oligo: | 5′ TCT GGA AAT TCT TCC AGA ATT GAT ACT GAC TGT |
| | Mismatched oligo: | 5′ GAT GAC TAT CTT TAA TGT ATG GAA AAT GAG AGC |
| 6. | Beacon-M/SDFI: | 5′ CGC GTG CCA GGT CTG CCT CTT CTA CGC G |
| | Matched oligo: | 5′ TCC CAG AAG AGG CAG ACC TGG GTG GGA |
| | Mismatched oligo: | 5′ GTC GCA GAA GAG GGA GAG GCG GGT GGG A |
| 7. | Beacon-W/SDF1: | 5′ CGA CGG AGG GGG GTG GGA TGC GTC G |
| | Matched oligo: | 5′ GAG ATG GGA GCC GGG TGT GCC TCT T |
| | Mismatched oLigo: | 5′ TGG AGA TGG GAG GGA GGT GTG CCT CTT CTG |
| 8. | Beacon-M/Factor V: | 5′ CGA CGT GGA GAG GGA AGG AAT AGC GTC G |
| | Matched oligo | 5′ GGT GTG TAT TCC TTG CCT GTC CAG GG |
| | Mismatched oligo: | 5′ TAG GTG TAT TGG TCG GGT GTG GAG GGA |
| 9. | Beacon-W/Factor V: | 5′ CGA CGT GTA TTC CTC GCG TGT GCG TCG |
| | Matched oligo. | 5′ GGG TGG AGA GGG GAG GAA TAG AGG T |
| | Mismatched oligo: | 5′ GGG TGG AGA GGG AAG GAA TAG AGA GG |
| 10. | Beacon-M/MTHFR: | 5′ CCG CTT GAT GAA ATG GAG TGG GGA GCG G |
| | Matched oligo: | 5′ GGT GTG TGG GGG AGT GGA TTT GAT GAT GAG G |
| | Mismatched ohgo: | 5′ GGT GTC TGG GGG AGG CGA TTT CAT GAT GAG G |
| 11. | Beacon-W/MTHFR: | 5′ CCG GTG GGG GAG CGG ATT TGA ACC GG |
| | Matched oligo: | 5′ GGT GAT GAT GAA ATG GGC TCG GGC AGA GAG G |
| | Mismatched oligo: | 5′ GGT GAT GAT GAA ATC GAG TGC CGG AGA GAG C |
| 12. | Beacon-M/FXIII: | 5′ CGC ACG GTT GAG GGG TTG GTG CCG TGC G |
| | Matched oligo: | 5′ GAG AGC ACC AAG CCC TGA AGC TAG AT |
| | Mismatched oligo | 5′ GAG AGC ACG AGG CCC TGA AGC TAG AT |
| 13. | Beacon-W/FXIII | 5′ GCG ACG GAG GAG GCC GTG AAG CCG TCG C |
| | Matched oligo: | 5′ GTG CGC TTG AGG GCG TGG TGA TGA |
| | Mismatched oligo: | 5′ GTG CGC TTG AGG GCT TGG TGA TGA |

Note: Bold letters in mismatched oligonucleotides indicate mismatched nucleotide.

### PCR Analysis

General description

The molecular beacons that demonstrated good target specificity by the melting curve analysis were used in PCR to test their ability in allele discrimination on human DNA. The beacons were tested by using both the cloned allele-specific control DNA and numerous human genomic DNA samples as the PCR templates. The human genomic DNA used includes the commercially available human genomic DNA (Sigma), 50 unrelated human genomic DNA samples and some genotype-validated human genomic DNA samples (Cenetron Diagnostics, Austin, Tex.). Briefly, the analysis began with the preparation of a PCR mixture that included the two allele-specific molecular beacons, the DNA template and other PCR reagents. The amplification was performed in the ABI7700 thermal cycler that automatically records the fluorescent signals generated during the annealing step of each PCR cycle. After a PCR run was complete, the fluorescence values were analyzed using the ABI7700 sequence detector software (v.6.0.1). The fluorescence values generated from the last PCR cycle (the endpoint fluorescence) were used to determine the allelic composition of a DNA sample. PCR amplification can be performed in a conventional thermal cycler (e.g., the Robocycler of Stratagene) and the ABI7700 thermal cycler is subsequently used to “read” the endpoint fluorescence. Alternatively, the threshold cycle (Ct) values can be used in determining the allelic composition of sample DNA. Threshold cycle is the PCR cycle at which the fluorescent signal is first detectable above background.

1. PCR Reactions

PCR analysis was carried out using both the cloned allele-specific control DNA and total genomic DNA as the templates. The natures of the five human gene mutations are described in Table 1. The sequences of the beacons are shown in Table 4. The general PCR protocol is described hereinabove. The specific choice of reagents (e.g., buffers and the templates) and that of the quantities of reagents are specified in the figure legends. Typical results of the analyses for the five pairs of allele-specific molecular beacons are shown below. The fluorescence values presented were those after background subtraction (i.e., baseline correction).

For all the beacons provided in the kits, we tested three PCR buffers, 5×Tris, 5×Tricine, 10×Taq (see above for buffer composition) for the specificity of amplification and for the fluorescence signal intensity. The three buffers resulted in high specific amplification of the CCR2, factor V and SDF1 (data not shown) fragments; Tricine buffer gave highest specificity for amplifying the CCR5 and MTHFR (data not shown) fragments. The same or higher fluorescent signal was generated with the Tricine buffer for three beacon systems (CCR2, CCR5, MTHFR). Therefore Tricine buffer is chosen for these kits. However, for reasons unclear, the fluorescence intensity was higher with the Tris buffer for the factor V beacons and with the Taq buffer for the SDF1 beacons (data not shown). These buffers are thus chosen for these kits, respectively.

2. PCR protocols

The typical PCR mixture consists of the following components:

| Working solution | Volume added | | Final conc. | |
|---|---|---|---|---|
| Allele 1 molecular beacon | 0.5 or 1 or 2 | μl | 0.1/0.2/0.4 | μM^{a} |
| (10 μM) | | | | |
| Allele 2 molecular beacon | 0.5 or 1 or 2 | μl | 0.1/0.2/0.4 | μM^{a} |
| (10 μM) | | | | |
| Forward primer (10 mM) | 1 or 2 | μl | 0.1/0.2 | mM |
| Reverse primer (10 mM) | 1 or 2 | μl | 0.1/0.2 | mM |
| dNTPs (20 mM mix) | 0.5 or 2 | μl | 0.05 or 0.2 | mM each |
| 5 x or 10 x PCR buffer^{b} | 5 or 10 | μl | 1 | x |
| DNA template | 2 to 5 | μl | Variable ^{c} | |
| Taq2000 (5 U/μl) | 0.5 | μl | 0.05 | U/μl |
| Total volume | 50 | μl | | |

a. One of the concentrations was chosen to achieve similar fluorescence values for both molecular beacons in the same kit. The concentration selected is described in the figure legends (see “Results”).

b. Three buffers were tested for all beacons (see bellow). The buffer that resulted in higher signal-to-background ratio and higher specificity of amplification was chosen for a given kit (see figure legends in “Results”):

Buffer A (5×Tris buffer): 250 mM KCl, 20 mM MgCl₂, 125 mM Tris, pH8.0.

Buffer B (5×Tricine buffer): 250 mM KCl, 20 mM MgCl₂, 375 mM Tricine, pH8.0.

Buffer C (10×Tris buffer): 500 mM KCl, 40 mM MgCl₂, 0.02% gelatin, 100 mM Tris, pH8.8.

Buffer D (10×Core buffer): 400 mM KCl, 30 mM MgCl₂, 1% Triton, 700 mM Tris, pH8.5

c. For cloned Allele 1 and Allele 2 DNA templates, 2 μl of a 10 pg/μl solution were added (final conc.: 20 pg/50 μl). For total human genomic DNA, 2 to 5 μl of the solutions with DNA concentrations ranging from 10 ng/μl to 100 ng/μl were added (final concentration: 10 to 500 ng/50 μl) (see “Results” for details). The concentration of the DNA samples was determined by UV absorbance at 260 nm using a spectrophotometer (Beckman DU600).

The typical thermal cycling condition consists of 95° C. for 2 minutes followed by 40 cycles of 95° C. for 30 seconds, 55° C. for 1 minute and 72° C. for 30 seconds. The PCR primers used for target amplification and the amplicon sizes are listed in Table 6.

TABLE 6
PCR Primers Used for Target Amplification and Detection

| Gene | Primers | SEQ ID NO | Amplicon Size (bp) |
|---|---|---|---|
| CCR2 | F: 5′-CTC TAC TCG CTG GTG TTC ATC | 13 | 103 |
| | R: 5′-GAG CAG GTA AAT GTC AGT CAA G | 14 | |
| CCR5 | F: 5′-CTT CAT TAC ACC TGC AGC TCT C | 15 | 108 (Wild Type) |
| | R: 5′-GAC AAG CAG CGG CAG GAC C | 16 | 76 (Mutant) |
| CCR5 | F: 5′-CCA GGA ATC ATC TTT ACC AG | 17 | 132 (Wild Type) |
| | R: 5′-CAG GAC CAG CCC CAA GAT GAC | 18 | 100 (Mutant) |
| SDF1 | F: 5′-CCC CTT CTC CAT CCA CAT | 19 | 52 |
| | R: 5′-TCC TCC CCT CCC AGA AGA | 20 | |
| | R: 5′-TGC TCC CCT CCC AGA AG | 21 | |
| Factor V | F: 5′-GAC ATC ATG AGA GAC ATC GC | 22 | 107 |
| | R: 5′-AGG TTA CTT CAA GGA CAA AAT AC | 23 | |
| MTHFR | F: 5′-ACT TGA AGG AGA AGG TGT CTG | 24 | 74 |
| | R: 5′-GAA GAA TGT GTC AGC CTC AAA G | 25 | |
| MTHFRF: | F: 5′-TGA CCT GAA GCA CTT GAA GGA | 26 | 68 |
| | R: 5′-CAA AGA AAA GCT GCG TGA TG | 27 | |
| Factor XIII | F: 5′-CCC AAT AAC TCT AAT GCA GCG | 28 | 91 |
| | R: 5′-TGC TCA TAC CTT GCA GGT TG | 29 | |

3. Analysis of Results

(1) Determination of allelic composition using endpoint fluorescence value

To determine the allelic composition of a sample DNA, three PCR control reactions were carried out in parallel with the amplification of sample DNA: A1 (allele 1), A2 (allele 2), and NT (no template). A1 and A2 controls contained either of the two cloned allele-specific DNA templates in addition to the beacons and PCR reagents. The endpoint fluorescence values (TET and FAM) generated from the sample DNA were compared to those generated from these controls. If the fluorescence values of the sample DNA were comparable with that of the A1 control (the wild type allele) by demonstrating high TET value and low FAM value, this sample was designated as a wild type homozygote (A1/A1). If the values were comparable with that of the A2 control (the mutant allele) by demonstrating high FAM value and low TET value, the sample was designated as a mutant homozygote (A2/A2). If the sample demonstrated intermediate-to-high values for both TET and FAM, it was designated as a heterozygote (A1/A2). The NT control was used as the reference for the background fluorescence generated by molecular beacons alone. A fourth control, A1/A2, could be used which contains a mixture of the two cloned allele-specific DNA templates in addition to the beacons and PCR reagents. This control is used as the reference for the presence of both alleles in the sample DNA (i.e., the heterozygote).

(2) Determination of allelic composition using threshold cycle value

Threshold cycle (Ct) is the cycle at which the fluorescent signal is first detectable above background. The Ct values are obtained by analyzing the real time fluorescence data using the ABI7700 sequence detection software. The allelic composition of a sample can be determined by plotting the Ct value generated by the wild-type specific beacon against the Ct value generated by the mutant specific beacon. In the current study, Ct values of 38-40 (40 is the maximum PCR cycle number used) are used to indicate the absence of a specific allele in the target DNA. Ct values between 20 to 30 are used to indicate the presence of a specific allele in the target DNA.

4. Gel electrophoresis

For the purpose of evaluating the specificity of target amplification, 5-μl aliquots of PCR products were separated by electrophoresis on a gel (a pre-cast 3:1 Nusieve/agarose gel supplied by FMC). The 100 bp DNA ladder (GIBCO/BRL) was used as the size marker. The gel image was recorded using the Eagle Eye II Still Video System (Stratagene).

### EXAMPLE I

1. Melting Curve Analysis

The analysis generated three melting curves for each beacon that represent three experimental conditions: the existence of the perfectly matched target, the existence of a mismatched target, and the existence of no target. For all molecular beacons described herein, the perfectly matched beacon/target hybrid generated higher fluorescence values than did the mismatched beacon/target hybrid at any temperature above 40° C. At the temperature below 40° C., some beacons generated equal amounts of fluorescence with both matched and mismatched beacon/target hybrids. For all beacons described herein, the fluorescence generated by the perfectly matched beacon/target hybrid endured at higher temperature. This finding is expected, as the perfectly matched beacon/target hybrids are thermally more stable than the mismatched beacon/target hybrids. The mismatched target used for the CCR5 beacon doesn't complement the sequence of the beacon and therefore no fluorescence signal was detected at any temperature. The complementary target sequence of the CCR5 beacon falls within the deleted region of the mutation (32-bp deletion).

### EXAMPLE 2

### CCR2 mutation

PCR analysis for the CCR2 allele-specific beacons was performed as follows.

20 pg of plasmid containing either the wild-type (W/W) or the mutant (M/M) DNA were used as the PCR templates in 50 μl reactions. They mimic either the wild-type or the mutant homozygous DNA (i.e., DNA that consists of two identical alleles). W/M indicates that 10 pg of each of the wild-type and the mutant plasmids were used as the template and it mimics the heterozygous DNA (i.e., DNA that consists of two different alleles). NT (no-template) indicates that TE buffer (10 mM Tris, 1 mM EDTA, pH8.0) was used instead of DNA template. Tricine buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The results show that only the correspondent fluorescence (background subtracted) was detected in the homozygous DNA (i.e., TET fluorescence in W/W and FAM fluorescence in M/M). As expected, both TET and FAM fluorescence were detected in the heterozygous DNA (W/M) and no fluorescence was detected in the no template control (NT). The intensity of the fluorescent signal detected in these samples is also as expected: higher intensity was detected in homozygous DNA and lower intensity in heterozygous DNA. Nine genotype-validated (for the CCR2 mutation) human genomic DNA samples were used as the PCR template (20 ng of DNA were used in 50 μl reactions). Tricine buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The results in were consistent with the predefined genotypes of these samples(Cindy WalkerPeach, Cenetron Diagnostics): 3 wild-type homozygous DNA (W/W), 3 mutant homozygous DNA (M/M) and 3 heterozygous DNA (W/M). Different amounts of a human genomic DNA sample (Sigma) were used as the PCR template to determine the effective range of target DNA concentration for the CCR2 beacon. Three different buffers (Tris, Tricine and Taq) were used to test the efficiency of PCR and that of fluorescence detection. 5 μl of PCR product was separated on the gel. The 100 bp ladder (GIBCO/BRL) was used as DNA size maker. The specific product (103 bp) was amplified with similar efficiency using three different buffers.

Screening of unknown genomic DNA samples for the CCR2 mutation were performed. The PCR templates used in this experiment are as follows: TE buffer (wells A1 through A8); 20 pg of plasmid containing the wild-type DNA (wells A9 through A12 and B1 through B4); 20 pg of plasmid containing the mutant DNA (wells B5 through B12); 10 pg of each of the wild-type and the mutant plasmids (wells C1 through C8); 50 unrelated human genomic DNA samples provided by Cenetron Diagnostics (20 ng each, D1 through H2); 9 genotype-validated human genomic DNA samples (20 ng each, H3 through H11). Tricine buffer was used in PCR. 0.4 μM (final concentration) of the mutant specific beacon and 0.2 μM (final concentration) of the wild-type specific beacon were used. The endpoint FAM fluorescence values generated by the mutant allele-specific beacon are shown. The same PCR reactions were examined for TET fluorescence generated by the wild-type allele-specific beacon. The experiment revealed 8 heterozygous DNA samples among the 50 unknown DNA samples (wells E2, E4, E5, E11, F1, F11, G4, G5). Among the 9 genotype validated homozygous wild-types (H4, H6, H8) and three were heterozygotes (H5, H9, H10). The results are in 100% concordance with that determined by Cenetron Diagnostics.

TABLE 7
Threshold cycle values for the CCR2 mutation detection

| | 1 | 2 | 3 | 4 | 5 | 6 | 7 | 8 | 9 | 10 | 11 | 12 |
|---|---|---|---|---|---|---|---|---|---|---|---|---|
| A | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 40 | A2: 40 | A2: 40 | A2: 40 | A2: 40 | A2: 40 | A2: 40 | A2: 40 | A2: 24.1 | A2: 24.1 | A2: 24.0 | A2: 23.8 |
| B | A1: 40 | A1: 40 | A1: 40 | A1: 40 | A1: 24.7 | A1: 24.7 | A1: 24.5 | A1: 24.4 | A1: 24.4 | A1: 24.4 | A1: 24.5 | A1: 24.2 |
| | A2: 24.3 | A2: 24.6 | A2: 24.3 | A2: 24.5 | A2: 40 | A2: 40 | A2: 40 | A2: 40 | A2: 40 | A2: 40 | A2: 40 | A2: 40 |
| C | A1: 24.7 | A1: 24.8 | A1: 24.6 | A1: 24.8 | A1: 24.6 | A1: 24.5 | A1: 24.4 | A1: 24.5 | A1: 40 | A1: 40 | A1: 40 | A1: 40 |
| | A2: 24.9 | A2: 25.1 | A2: 25.0 | A2: 25.2 | A2: 24.9 | A2: 24.9 | A2: 24.8 | A2: 24.8 | A2: 40 | A2: 40 | A2: 40 | A2: 40 |
| D | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 28.8 | A2: 28.1 | A2: 28.3 | A2: 2 92 | A2: 28.9 | A2: 28.5 | A2: 28.0 | A2: 27.5 | A2: 28.1 | A2: 28.1 | A2: 29.0 | A2: 28.0 |
| E | A1: 40 | A1: 28.9 | A1: 40 | A1: 29.1 | A1: 28.3 | A1: 40 | A1: 40 | A1: 40 | A1: 40 | A1: 40 | A1: 28.5 | A1: 40 |
| | A2: 28.4 | A2: 29.3 | A2: 27.1 | A2: 29.3 | A2: 29.0 | A2: 27.5 | A2: 27.5 | A2: 27.4 | A2: 28.0 | A2: 27.6 | A2: 28.5 | A2: 27.9 |
| F | A1: 28.7 | A1: 40 | A1: 40 | A1: 40 | A1: 40 | A1: 40 | A1: 40 | A1: 40 | A1: 40 | A1: 40 | A1: 28.3 | A1: 40 |
| | A2: 29.1 | A2: 28.0 | A2: 28.4 | A2: 27.9 | A2: 27.5 | A2: 27.3 | A2: 28.0 | A2: 27 6 | A2: 28.1 | A2: 28.2 | A2: 28.3 | A2: 28.1 |
| G | A1: 40 | A1: 40 | A1: 40 | A1: 29.0 | A1: 29.1 | A1: 40 | A1: 40 | A1: 40 | A1: 40 | A1: 40 | A1: 40 | A1: 40 |
| | A2: 27.8 | A2: 28.2 | A2: 27.8 | A2: 29.1 | A2: 29 4 | A2: 27.4 | A2: 27.7 | A2: 27.9 | A2: 28.2 | A2: 28.3 | A2: 28 2 | A2: 28.6 |
| H | A1: 40 | A1: 40 | A1: 27.0 | A1: 40 | A1: 28.4 | A1: 40 | A1: 28.0 | A1: 40 | A1: 28.5 | A1: 28.3 | A1: 27.3 | A1: 40 |
| | A2: 28.6 | A2: 28 2 | A2: 40 | A2: 27.5 | A2: 28.3 | A2: 27.8 | A2: 40 | A2: 28.1 | A2: 28.4 | A2: 28.3 | A2: 40 | A2: 40 |

AD(EP)-15 (CCR2). A1 = mutant allele (FAM signal). A2 = wild-type allele (TET signal).

The results for the control samples were as expected: Maximum Ct value (40) for both the mutant (A1) and wild-type (A2) alleles were obtained in no template controls (wells A1-A8); Maximum Ct value for the mutant allele and lower Ct values (24-25) for wild-type allele were obtained in the wild-type only controls (wells A9-A12, B1-B4); Maximum Ct value for the wild-type allele and lower Ct values (24-25) for the mutant allele were obtained in the mutant only controls (wells B5-B12); Lower Ct values (24-26) for both the mutant and the wild-type alleles were obtained in the heterozygote controls (wells C1-C8). The analysis revealed 8 heterozygous DNA samples among the 50 unknown DNA samples (wells E2, E4, E5, E11, F1, F11, G4, G5). Among the 9 genotype validated samples, three homozygous mutants (wells H3, H7, H11), three homozygous wild types (H4, H6, H8) and three heterozygotes (H5, H9, H10) were found.

### EXAMPLE 3

### CCR5 Mutation

20 pg of plasmid containing either the wild-type (W/W) or the mutant (M/M) DNA were used as the PCR templates in 50 μl reactions. They mimic either the wild-type or the mutant homozygous DNA. W/M indicates that 10 pg of each of the wild-type and the mutant plasmids were used as the template and it mimics the heterozygous DNA. NT (no-template) indicates that TE buffer was used instead of DNA template. Tricine buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The results show that only the correspondent fluorescence (background subtracted) was detected in the homozygous DNA (i.e., TET fluorescence in W/W and FAM fluorescence in M/M). As expected, both TET and FAM fluorescence were detected in the heterozygous DNA (W/M) and no fluorescence was detected in the no template control (NT). The intensity of the fluorescent signal detected in these samples was also as expected: higher intensity was detected in homozygous DNA and lower amount in heterozygous DNA. Three different buffers (Tris, Tricine and Taq) were used in PCR and comparable results (endpoint fluorescence value and Ct value) for the CCR5 beacon were obtained (not shown). PCR was carried out using 20 pg of plasmids containing the wild-type DNA (W), the mutant DNA(M), or 10 pg of each of the two plasmids (W/M) as the template. 5 μl of PCR product was separated on the gel. The 100 bp ladder (GIBCO/BRL) was used as DNA size maker. The size of the wild type-allele specific product is 108 bp and that of the mutant-specific product is 76 bp.

PCR templates used in this experiment are as follows: TE buffer (wells A1 through A8); 20 pg of plasmid containing the wild-type DNA (wells A9 through A12 and B1 through B4); 20 pg of plasmid containing the mutant DNA (wells B5 through B12); 10 pg of each of the wild-type and the mutant plasmids (wells C1 through C8); 50 unrelated human genomic DNA samples provided by Cenetron Diagnostics (20 ng each, D1 through H2); 9 genotype-validated (for the CCR5 mutation) human genomic DNA samples (20 ng each, H3 through H11). Tricine buffer was used in PCR. 0.4 μM (final concentration) of the mutant specific beacon and 0.2 μM (final concentration) of the wild-type specific beacon were used. The endpoint FAM fluorescence values generated by the mutant allele-specific beacon are shown. The same PCR reactions were examined for TET fluorescence generated by the wild-type allele-specific beacon. The TET fluorescence values were unexpectedly high in the no template controls (wells A1-A8), suggesting that these samples were contaminated by the wild-type control plasmid (see Table 8). The experiment detected 9 heterozygous DNA samples among the 50 unknown DNA samples (wells D1, D2, D7, E4, E12, F4, F7, G2, G11). Among the 9 genotype validated samples, one was found to be mutant homozygote (H7) and one was heterozygote (H4). The results are in 100% concordance with that determined by Cenetron Diagnostics.

TABLE 8
Threshold cycle values for CCR5 mutation detection

| | 1 | 2 | 3 | 4 | 5 | 6 | 7 | 8 | 9 | 10 | 11 | 12 |
|---|---|---|---|---|---|---|---|---|---|---|---|---|
| A | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 32.6 | A2: 32.1 | A2: 32.0 | A2: 32.3 | A2: 31 8 | A2: 32.6 | A2: 32.1 | A2: 32.8 | A2: 25.4 | A2: 25 6 | A2: 25 6 | A2: 25 5 |
| B | A1: 40 | A1: 40 | A1: 40 | A1: 40 | A1: 26 7 | A1: 26.6 | A1: 25.7 | A1: 26.5 | A1: 25.5 | A1: 25.0 | A1: 25.4 | A1: 25.0 |
| | A2: 25.5 | A2: 25.2 | A2: 25.3 | A2: 25.3 | A2: 40 | A2: 40 | A2: 40 | A2: 40 | A2: 40 | A2: 40 | A2: 40 | A2: 40 |
| C | A1: 26.8 | A1: 27.8 | A1: 26.2 | A1: 27.0 | A1: 26.9 | A1: 27.1 | A1: 27.0 | A1: 26.1 | A1: 40 | A1: 40 | A1: 40 | A1: 40 |
| | A2: 25.4 | A2: 25.6 | A2: 24.3 | A2: 25.3 | A2: 24.9 | A2: 25.7 | A2: 26.1 | A2: 25.6 | A2: 40 | A2: 40 | A2: 40 | A2: 40 |
| D | A1: 33.3 | A1: 31.8 | A1: 40 | A1: 40 | A1: 40 | A1: 40 | A1: 31.0 | A1: 40 | A1: 40 | A1: 40 | A1: 40 | A1: 40 |
| | A2: 30.5 | A2: 30.0 | A2: 28.4 | A2: 28.7 | A2: 28.0 | A2: 29.6 | A2: 29.5 | A2: 28.6 | A2: 28.6 | A2: 29.6 | A2: 29.1 | A2: 30.0 |
| E | A1: 40 | A1: 40 | A1: 40 | A1: 29.9 | A1: 40 | A1: 40 | A1: 40 | A1: 40 | A1: 40 | A1: 40 | A1: 40 | A1: 30.3 |
| | A2: 29.0 | A2: 28.1 | A2: 25.7 | A2: 28.2 | A2: 27.5 | A2: 27.6 | A2: 27.9 | A2: 27.8 | A2: 28.3 | A2: 28.3 | A2: 28.9 | A2: 30.0 |
| F | A1: 40 | A1: 40 | A1: 40 | A1: 31.7 | A1: 40 | A1: 40 | A1: 31.1 | A1: 40 | A1: 40 | A1: 40 | A1: 40 | A1: 40 |
| | A2: 27.7 | A2: 28.3 | A2: 28.3 | A2: 29.6 | A2: 28.3 | A2: 28.1 | A2: 30.0 | A2: 28.4 | A2: 28.7 | A2: 28.7 | A2: 28.4 | A2: 28.8 |
| G | A1: 40 | A1: 30.8 | A1: 40 | A1: 40 | A1: 40 | A1: 40 | A1: 40 | A1: 40 | A1: 40 | A1: 40 | A1: 29.6 | A1: 40 |
| | A2: 27.6 | A2: 29.0 | A2: 27.6 | A2: 28.2 | A2: 28.3 | A2: 27.3 | A2: 28.3 | A2: 28.1 | A2: 27.1 | A2: 28.2 | A2: 28.8 | A2: 28.2 |
| H | A1: 40 | A1: 40 | A1: 40 | A1: 29.5 | A1: 40 | A1: 40 | A1: 24.4 | A1: 40 | A1: 40 | A1: 40 | A1: 40 | A1: 40 |
| | A2: 26.9 | A2: 28.3 | A2: 26.4 | A2: 27.9 | A2: 27.2 | A2: 26.6 | A2: 40 | A2: 26.4 | A2: 26.7 | A2: 23.0 | A2: 25.9 | A2: 40 |

AD(EP)-16 (CCR5). A1 = mutant allele (FAM signal). A2 = wild-type allele (TET signal).

The results for the control samples were as expected except for the no template controls: Maximum Ct value (40) for the mutant (A1) allele but lower Ct values (˜32) for the wild-type allele (A2) were obtained in no template controls (wells A1-A8), suggesting that these samples were contaminated by the wild-type control plasmid; Maximum Ct value for the mutant allele and lower Ct values (˜25) for the wild-type allele were obtained in the wild-type only controls (wells A9-A12, B1-B4); Maximum Ct value for the wild-type allele and lower Ct values (25-27) for the mutant allele were obtained in the mutant only controls (wells B5-B12); Lower Ct values (24-27) for both the mutant and the wild-type alleles were obtained in the heterozygote controls (wells C1-C8). The analysis revealed 9 heterozygous DNA samples among the 50 unknown DNA samples (wells D1, D2, D7, E4, E12, F4, F7, G2, G11). Among the 9 genotype validated samples, one was found to be mutant homozygote (H7) and one was found to be heterozygote (H4).

### EXAMPLE 4

### SDF1 Mutation

The results of PCR analysis for the SDF1 allele-specific beacons are as follows. 20 pg of plasmid containing either the wild-type (W/W) or the mutant (M/M) DNA were used as the PCR templates in 50 μl reactions. W/M indicates that 10 pg of each of the wild-type and the mutant plasmids were used as the templates. NT (no-template) indicates that TE buffer was used instead of DNA template. Taq buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The results show that only the correspondent fluorescence (background subtracted) was detected in the homozygous DNA (i.e., TET fluorescence in W/W and FAM fluorescence in M/M). As expected, both TET and FAM fluorescence were detected in the heterozygous DNA (W/M) and no fluorescence was detected in the no template control (NT). The intensity of the fluorescent signal detected in these samples is also as expected: higher intensity was detected in homozygous DNA and lower intensity in heterozygous DNA. 5 μl of PCR product from above experiment was separated on the gel. The 100 bp ladder (GIBCO/BRL) was used as DNA size maker. The size of the specific product is 52 bp.

The results of screening of unknown genomic DNA samples for the SDF1 mutation are as follows. PCR templates used in this experiment are as follows: TE buffer (wells A1 through A4); 20 pg of plasmid containing the wild-type DNA (wells A5 through A8); 20 pg of plasmid containing the mutant DNA (wells A9 through A12); 10 pg of each of the wild-type and the mutant plasmids (wells B1 through B4); 50 unrelated human genomic DNA samples provided by Cenetron Diagnostics (20 ng each, C1 through G2). Taq buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The FAM fluorescence values were unexpectedly high in two of the no template controls (wells A1 and A2), suggesting that these samples were contaminated by the mutant control plasmid. The experiment detected three mutant homozygous DNA samples (wells C7, D3, F3) and 15 heterozygous DNA samples (wells C2, C5, C6, C10, C12, D2, D12, E1, E6, E8, E9, E10, F1, F7, F9) among the 50 unknown DNA samples. The results are in 100% concordance with that determined by Cenetron Diagnostics.

TABLE 9
Threshold cycle values for SDF1 mutation detection

| | 1 | 2 | 3 | 4 | 5 | 6 | 7 | 8 | 9 | 10 | 11 | 12 |
|---|---|---|---|---|---|---|---|---|---|---|---|---|
| A | A1: 36.0 | A1: 37.1 | A1: 40 | A1: 40 | A1: 40 | A1: 40 | A1: 40 | A1: 40 | A1: 24.0 | A1: 24.2 | A1: 23.9 | A1: 23.9 |
| | A2: 40 | A2: 40 | A2: 40 | A2: 40 | A2: 24.1 | A2: 24 0 | A2: 23.9 | A2: 23.7 | A2: 40 | A2: 40 | A2: 40 | A2: 40 |
| B | A1: 25.9 | A1: 25.8 | A1: 25.4 | A1: 25.3 | A1: 40 | A1: 40 | A1: 40 | A1: 40 | A1: 40 | A1: 40 | A1: 40 | A1: 40 |
| | A2: 24.4 | A2: 24.5 | A2: 24.2 | A2: 24.2 | A2: 40 | A2: 40 | A2: 40 | A2: 40 | A2: 40 | A2: 40 | A2: 40 | A2: 40 |
| C | A1: 40 | A1: 30.5 | A1: 40 | A1: 40 | A1: 30.4 | A1: 30.2 | A1: 29 | A1: 40 | A1: 40 | A1: 30.5 | A1: 40 | A1: 29.4 |
| | A2: 28.2 | A2: 29.3 | A2: 28.0 | A2: 28.3 | A2: 29.1 | A2: 29.3 | A2: 40 | A2: 28.0 | A2: 27.9 | A2: 29.2 | A2: 27.9 | A2: 28.6 |
| D | A1: 40 | A1: 30.4 | A1: 28.1 | A1: 40 | A1: 40 | A1: 40 | A1: 40 | A1: 40 | A1: 40 | A1: 40 | A1: 40 | A1: 32.2 |
| | A2: 28.4 | A2: 29.4 | A2: 40 | A2: 28.3 | A2: 27.9 | A2: 27.5 | A2: 27.6 | A2: 27.2 | A2: 27.9 | A2: 27.6 | A2: 27 3 | A2: 29 4 |
| E | A1: 30.1 | A1: 40 | A1: 40 | A1: 40 | A1: 40 | A1: 29.7 | A1: 40 | A1: 29 9 | A1: 29 9 | A1: 29.8 | A1: 40 | A1: 40 |
| | A2: 29.3 | A2: 27.8 | A2: 28.1 | A2: 27.7 | A2: 27.7 | A2: 28.3 | A2: 27.4 | A2: 28.7 | A2: 28.8 | A2: 28.5 | A2: 27 4 | A2: 27.9 |
| F | A1: 30.2 | A1: 40 | A1: 28.2 | A1: 40 | A1: 40 | A1: 40 | A1: 29.4 | A1: 40 | A1: 30.0 | A1: 40 | A1: 40 | A1: 40 |
| | A2: 29.1 | A2: 28.1 | A2: 40 | A2: 27.9 | A2: 28.0 | A2: 27.6 | A2: 28.4 | A2: 27.4 | A2: 29.1 | A2: 27.7 | A2: 27.7 | A2: 27.8 |
| G | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 27.7 | A2: 27.9 | A2: 40 | A2: 40 | A2: 40 | A2: 40 | A2: 40 | A2: 40 | A2: 40 | A2: 40 | A2: 40 | A2: 40 |
| H | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 40 | A2: 40 | A2: 40 | A2: 40 | A2: 40 | A2: 40 | A2: 40 | A2: 40 | A2: 40 | A2: 40 | A2: 40 | A2: 40 |

AD(EP)-19(SDF1). A1 = mutant allele (FAM signal). A2 = wild-type allele (TET signal).

The results for the control samples were as expected: Maximum Ct values for both the mutant (A1) and wild-type (A2) alleles (except the values of FAM in A1 and A2) were obtained in no template controls (wells A1-A4); Maximum Ct value for the mutant allele and lower Ct values (˜24) for wild-type allele were obtained in the wild-type only controls (wells A5-A8); Maximum Ct value for the wild-type allele and lower Ct values (˜24) for the mutant allele were obtained in the mutant only controls (wells A9-A12); Lower Ct values (24-25) for both the mutant and the wild-type alleles were obtained in the heterozygote controls (wells B1-B4). The analysis revealed three homozygous mutant3 (wells C7, D3, F3) and 15 heterozygous DNA samples (wells C2, C5, C6, C10, C12, D2, D12, E1, E6, E8, E9, E10, F1, F7, F9) among the 50 unknown DNA samples.

### EXAMPLE 5

### Factor V Mutation

The results of PCR analysis for the Factor V allele-specific beacons are as follows. 20 pg of plasmid containing either the wild-type (W/W) or the mutant (M/M) DNA were used as the PCR templates in 50 μl reactions. They mimic either the wild-type or the mutant homozygous DNA. W/M indicates that 10 pg of each of the wild-type and the mutant plasmids were used as the template and it mimics the heterozygous DNA. NT (no-template) indicates that 1×TE buffer was used instead of DNA template. Tris buffer was used in PCR. 0.4 μM (final concentration) of the mutant specific beacon and 0.2 μM (final concentration) of the wild-type specific beacon were used. FAM fluorescence generated by the mutant allele-specific beacon was shown. The same PCR reactions were examined for TET fluorescence generated by the wild-type allele-specific beacon. The results show that only the correspondent fluorescence (background subtracted) was detected in the homozygous DNA (i.e., TET fluorescence in W/W and FAM fluorescence in M/M). As expected, both TET and FAM fluorescence were detected in the heterozygous DNA (W/M) and no fluorescence was detected in the no template control (NT). The intensity of the fluorescent signal detected in these samples is also as expected: higher intensity was detected in homozygous DNA and lower intensity in heterozygous DNA. Six genotype-validated (for the factor V mutation) human genomic DNA samples, provided by Cenetron Diagnostics (Austin, Tex.), were used as the PCR template (20 ng of DNA were used in 50 μl reactions). Tris buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions were examined for TET fluorescence generated by the wild-type allele-specific beacon. The results were consistent with the predefined genotypes of these samples with 2 homozygous wild types (W/W), 2 homozygous mutants (M/M) and 2 heterozygotes (W/M). Different amounts of a human genomic DNA sample (Sigma) were used as the PCR template to determine the effective range of target DNA concentration for the factor V beacon. Three different buffers (Tris, Tricine and Taq) were used to test the efficiency of PCR and that of fluorescence detection. 5 μl of PCR product was separated on the gel. The 100 bp ladder (GIBCO/BRL) was used as DNA size maker. The specific product (107 bp) was amplified with similar efficiency using three different buffers.

The results of screening of unknown genomic DNA samples for the FV mutation are as follows. The PCR templates used in this experiment are as follows: 1×TE buffer (wells A1 through A4); 20 pg of plasmid containing the wild-type DNA (wells A5 through A8); 20 pg of plasmid containing the mutant DNA (wells A9 through A12); 10 pg of each of the wild-type and the mutant plasmids (wells B1 through B4); 50 unrelated human genomic DNA samples provided by Cenetron Diagnostics (20 ng each, C1 through G2); 6 genotype-validated (for the FV mutation) human genomic DNA samples (20 ng each, G3 through G8); 9 genotype-validated (for the CCR2 mutation) human genomic DNA samples (wells G9 through H5). Tris buffer was used in PCR. 0.4 μM (final concentration) of the mutant specific beacon and 0.2 μM (final concentration) of the wild-type specific beacon were used. FAM fluorescence generated by the mutant allele-specific beacon is shown. The same PCR reactions in A were examined for TET fluorescence generated by the wild-type allele-specific beacon. The experiment detected 5 heterozygous DNA samples among the 50 unknown DNA samples (wells C4, C8, D11, E7, F2). Among the 6 genotype validated samples (for the FV mutation), two were found to be mutant homozygotes (wells G3, G4), two were wild-type homozygotes (G7, G8) and two were heterozygotes (G5, G6). Among the 9 genotype validated samples (for the CCR2 mutation), no FV mutant was detected. The results are in 100% concordance with that determined by Cenetron Diagnostics.

TABLE 10
Threshold cycle values for factor V mutation detection

| | 1 | 2 | 3 | 4 | 5 | 6 | 7 | 8 | 9 | 10 | 11 | 12 |
|---|---|---|---|---|---|---|---|---|---|---|---|---|
| A | A1: 40 | A1: 40 | A1: 40 | A1: 40 | A1: 40 | A1: 40 | A1: 40 | A1: 40 | A1: 27.7 | A1: 27.9 | A1: 27.9 | A1: 28.2 |
| | A2: 40 | A2: 39.1 | A2: 39.9 | A2: 40 | A2: 27.4 | A2: 27.1 | A2: 27.6 | A2: 27.5 | A2: 40 | A2: 40 | A2: 40 | A2: 40 |
| B | A1: 29.4 | A1: 29.9 | A1: 40 | A1: 29.5 | A1: 40 | A1: 40 | A1: 40 | A1: 40 | A1: 40 | A1: 40 | A1: 40 | A1: 40 |
| | A2: 28.8 | A2: 28.4 | A2: 27.4 | A2: 28.8 | A2: 40 | A2: 39.5 | A2: 40 | A2: 38.3 | A2: 40 | A2: 40 | A2: 40 | A2: 40 |
| C | A1: 40 | A1: 40 | A1: 40 | A1: 31.3 | A1: 40 | A1: 40 | A1: 40 | A1: 31.4 | A1: 40 | A1: 40 | A1: 40 | A1: 40 |
| | A2: 28.0 | A2: 28.3 | A2: 27.2 | A2: 29.7 | A2: 26.6 | A2: 27.4 | A2: 28.8 | A2: 30.2 | A2: 28.3 | A2: 28.6 | A2: 28.7 | A2: 29.6 |
| D | A1: 40 | A1: 40 | A1: 40 | A1: 40 | A1: 40 | A1: 40 | A1: 40 | A1: 40 | A1: 40 | A1: 40 | A1: 30.6 | A1: 40 |
| | A2: 27.2 | A2: 25.9 | A2: 28.3 | A2: 276 | A2: 27.6 | A2: 28.0 | A2: 28.7 | A2: 26.3 | A2: 28.3 | A2: 29.1 | A2: 28.4 | A2: 30.0 |
| E | A1: 40 | A1: 40 | A1: 40 | A1: 40 | A1: 40 | A1: 40 | A1: 32.4 | A1: 40 | A1: 40 | A1: 40 | A1: 40 | A1: 40 |
| | A2: 26.7 | A2: 27.6 | A2: 28.1 | A2: 26.9 | A2: 25.0 | A2: 28.1 | A2: 29.0 | A2: 26.6 | A2: 27.7 | A2: 27.2 | A2: 27 3 | A2: 28.2 |
| F | A1: 40 | A1: 40 | A1: 30.4 | A1: 40 | A1: 40 | A1: 40 | A1: 40 | A1: 40 | A1: 40 | A1: 40 | A1: 40 | A1: 40 |
| | A2: 27.7 | A2: 28.1 | A2: 26.2 | A2: 28.1 | A2: 28.6 | A2: 27 2 | A2: 28.6 | A2: 26.7 | A2: 29.0 | A2: 28.1 | A2: 27.8 | A2: 26.4 |
| G | A1: 40 | A1: 40 | A1: 28.0 | A1: 28.0 | A1: 29.2 | A1: 28.5 | A1: 40 | A1: 40 | A1: 40 | A1: 40 | A1: 40 | A1: 40 |
| | A2: 27.0 | A2: 26.6 | A2: 40 | A2: 40 | A2: 27.8 | A2: 27.1 | A2: 28.3 | A2: 27.7 | A2: 26.9 | A2: 27.5 | A2: 27.6 | A2: 27.9 |
| H | A1: 40 | A1: 40 | A1: 40 | A1: 29.5 | A1: 40 | A1: 40 | A1: 24.4 | A1: 40 | A1: 40 | A1: 40 | A1: 40 | A1: 40 |
| | A2: 26.7 | A2: 27.1 | A2: 26.8 | A2: 26.4 | A2: 25.7 | A2: 40 | A2: 40 | A2: 40 | A2: 40 | A2: 40 | A2: 40 | A2: 40 |

AD(EP)-11 (FV). A1 = mutant allele (FAM signal). A2 = wild-type allele (TET signal).

The results for the control samples were as expected: Maximum Ct value for both the mutant (A1) and wild-type (A2) alleles were obtained in no template controls (wells A1-A4); Maximum Ct value for the mutant allele and lower Ct values (˜27) for wild-type allele were obtained in the wild-type only controls (wells A5-A8); Maximum Ct value for the wild-type allele and lower Ct value for the mutant allele were obtained in the mutant only controls (wells A9-A12); Lower Ct values (28-29) for both the mutant and the wild-type alleles were obtained in the heterozygote controls (wells B1-B4). The analysis revealed 5 heterozygous DNA samples among the 50 unknown DNA samples (wells C4, C8, D11, E7, F2). Among the 6 genotype validated samples (for the FV mutation), two were found to be homozygous mutants (wells G3, G4), two were homozygous wild-types (G7, G8) and two were heterozygotes (G5, G6). Among the 9 genotype validated samples (for the CCR2 mutation) (G9-G12, H1-H5), no FV mutant was detected.

### EXAMPLE 6

### MTHFR Analysis

The results of PCR analysis for the MTHFR allele-specific beacons was as follows. 20 pg of plasmid containing either the wild-type (W/W) or the mutant (M/M) DNA were used as the PCR templates in 50 μl reactions. They mimic either the wild-type or the mutant homozygous DNA. W/M indicates that 10 pg of each of the wild-type and the mutant plasmids were used as the template and it mimics the heterozygous DNA. NT (no-template) indicates that TE buffer was used instead of DNA template. Tricine buffer was used in PCR. 0.2 μM (final concentration) of the mutant specific beacon and 0.2 μM (final concentration) of the wild-type specific beacon were used. FAM fluorescence generated by the mutant allele-specific beacon was shown. The same PCR reactions were examined for TET fluorescence generated by the wild-type allele-specific beacon. The results show that only the correspondent fluorescence (background subtracted) was detected in the homozygous DNA (i.e., TET fluorescence in W/W and FAM fluorescence in M/M). As expected, both TET and FAM fluorescence were detected in the heterozygous DNA (W/M) and no fluorescence was detected in the no template control (NT). The intensity of the fluorescent signal detected in these samples was also as expected: higher intensity was detected in homozygous DNA and lower intensity in heterozygous DNA. 5 μl of PCR product from above experiment was separated on the gel. The 100 bp ladder (GIBCO/BRL) was used as DNA size maker. The size of the specific product is 74 bp. The type of PCR templates used are indicated: the wild-type DNA (W), the mutant DNA(M), both the wild-type and the mutant DNA (W/M) and no template (NT).

The screening of unknown genomic DNA samples for the MTHFR mutation was as follows. PCR templates used in this experiment are as follows: TE buffer (wells A1 through A4); 20 pg of plasmid containing the wild-type DNA (wells A5 through A8); 20 pg of plasmid containing the mutant DNA (wells A9 through A12); 10 pg of each of the wild-type and the mutant plasmids (wells B1 through B4); 50 unrelated human genomic DNA samples provided by Cenetron Diagnostics (20 ng each, C1 through G2). Tricine buffer was used in PCR. 0.2 μM (final concentration) of the mutant specific beacon and 0.2 μM (final concentration) of the wild-type specific beacon were used. FAM fluorescence generated by the mutant allele-specific beacon is shown. The same PCR reactions in were examined for TET fluorescence generated by the wild-type allele-specific beacon. The experiment detected two mutant homozygous DNA samples (wells D2, F1) and 30 heterozygous DNA samples (wells C1, C4, C6, C9-12, D3, D4, D8-12, E1, E3, E6, E7, E9, E11, E12, F2-4, F6, F9, F11, F12, G1, G2) among the 50 unknown DNA samples. The results are in 100% concordance with that determined by Cenetron Diagnostics.

TABLE 11
Threshold cycle values for MHTFR mutation detection

| | 1 | 2 | 3 | 4 | 5 | 6 | 7 | 8 | 9 | 10 | 11 | 12 |
|---|---|---|---|---|---|---|---|---|---|---|---|---|
| A | A1: 40 | A1: 40 | A1: 40 | A1: 40 | A1: 40 | A1: 40 | A1: 40 | A1: 40 | A1: 22.2 | A1: 22.3 | A1: 22.2 | A1: 22.2 |
| | A2: 38.3 | A2: 39.4 | A2: 38.9 | A2: 38.8 | A2: 23.5 | A2: 23.4 | A2: 23.1 | A2: 23.3 | A2: 40 | A2: 40 | A2: 40 | A2: 40 |
| B | A1: 23.9 | A1: 24.1 | A1: 23.6 | A1: 23.9 | A1: 40 | A1: 40 | A1: 40 | A1: 40 | A1: 40 | A1: 40 | A1: 40 | A1: 40 |
| | A2: 24.8 | A2: 25.0 | A2: 24.6 | A2: 24.8 | A2: 40 | A2: 40 | A2: 40 | A2: 40 | A2: 40 | A2: 40 | A2: 40 | A2: 40 |
| C | A1: 32.6 | A1: 40 | A1: 40 | A1: 31.5 | A1: 40 | A1: 31.4 | A1: 40 | A1: 40 | A1: 30.4 | A1: 31.2 | A1: 30.8 | A1: 30.8 |
| | A2: 33.0 | A2: 31.3 | A2: 30.0 | A2: 31.9 | A2: 30.1 | A2: 31.7 | A2: 31.1 | A2: 30.3 | A2: 31.2 | A2: 31.7 | A2: 31.1 | A2: 31.5 |
| D | A1: 40 | A1: 30.5 | A1: 30.5 | A1: 31.1 | A1: 40 | A1: 40 | A1: 40 | A1: 29.4 | A1: 29.9 | A1: 29.6 | A1: 29.4 | A1: 28.8 |
| | A2: 31.4 | A2: 40 | A2: 31.1 | A2: 31.6 | A2: 28.9 | A2: 29.1 | A2: 29.7 | A2: 30.3 | A2: 31.1 | A2: 30.3 | A2: 30.7 | A2: 29.4 |
| E | A1: 30.8 | A1: 40 | A1: 31.5 | A1: 40 | A1: 40 | A1: 30.4 | A1: 30.4 | A1: 40 | A1: 30.7 | A1: 40 | A1: 30.8 | A1: 31.2 |
| | A2: 31.8 | A2: 29.7 | A2: 31.9 | A2: 30.3 | A2: 30.5 | A2: 30.7 | A2: 30.8 | A2: 29.4 | A2: 31.1 | A2: 30.0 | A2: 31.1 | A2: 31.9 |
| F | A1: 29.0 | A1: 30.8 | A1: 31.1 | A1: 31.6 | A1: 40 | A1: 31.2 | A1: 40 | A1: 40 | A1: 31.3 | A1: 40 | A1: 30.9 | A1: 31.1 |
| | A2: 40 | A2: 31.4 | A2: 31.5 | A2: 32.1 | A2: 31.4 | A2: 32.0 | A2: 30.7 | A2: 30.4 | A2: 31.8 | A2: 30.8 | A2: 32.0 | A2: 32.2 |
| G | A1: 32.1 | A1: 31.1 | A1: 40 | A1: 40 | A1: 40 | A1: 40 | A1: 40 | A1: 40 | A1: 40 | A1: 40 | A1: 40 | A1: 40 |
| | A2: 32.5 | A2: 31.9 | A2: 40 | A2: 40 | A2: 40 | A2: 40 | A2: 40 | A2: 40 | A2: 40 | A2: 40 | A2: 40 | A2: 40 |
| H | A1: 40 | A1: 40 | A1: 40 | A1: 40 | A1: 40 | A1: 40 | A1: 40 | A1: 40 | A1: 40 | A1: 40 | A1: 40 | A1: 40 |
| | A2: 40 | A2: 40 | A2: 40 | A2: 40 | A2: 40 | A2: 40 | A2: 40 | A2: 40 | A2: 40 | A2: 40 | A2: 40 | A2: 40 |

AD(EP)-20 (MTHFR). A1 = mutant allele (FAM signal). A2 = wild-type allele (TET signal).

The results for the control samples were as expected: Maximum or close to maximum Ct value for both the mutant (A1) and wild-type (A2) alleles were obtained in no template controls (wells A1-A4); Maximum Ct value for the mutant allele and lower Ct values (˜23) for wild-type allele were obtained in the wild-type only controls (wells A5-A8); Maximum Ct value for the wild-type allele and lower Ct values (˜22) for the mutant allele were obtained in the mutant only controls (wells A9-A12); Lower Ct values (24-25) for both the mutant and the wild-type alleles were obtained in the heterozygote controls (wells B1-B4). The analysis revealed two homozygous mutants (wells D2, F1) and 30 heterozygotes (wells C1, C4, C6, C9-12, D3, D4, D8-12, E1, E3, E6, E7, E9, E11, E12, F2-4, F6, F9, F11, F12, G1, G2) among the 50 unknown DNA samples.

### EXAMPLE 7

### Factor XIII Mutation

The results of PCR analysis for the Factor XIII allele-specific beacons was as follows. 50-100 ng of human genomic DNA (gDNA) that was homozygous for the wild-type (W/W; indicated as GG) Factor XIII allele was used as PCR templates in 50 μl reactions. Also in pg 1 of plasmid containing the homozygous wild-type (W/W; indicated as GG) DNA mixed in 20 ng mouse gDNA was used as a PCR template. The plasmid mixed into mouse gDNA mimics the wild-type homozygous DNA. NTC (no-template control) indicates that 1×TE buffer was used instead of DNA template. Tricine buffer was used in PCR. 0.2 μM (final concentration) of the wild-type specific beacon was used. FAM fluorescence generated by the wild-type allele-specific beacon is shown. The results of PCR are shown in which the template DNAs were either 50-100 ng of human gDNA that was homozygous for the mutant (M/M; indicated as TT) Factor XIII allele or 0.5 pg of plasmid containing the mutant (M/M; indicated as TT) DNA mixed in 20 ng mouse gDNA. The plasmid mixed into mouse gDNA mimics the mutant homozygous DNA. NTC (no-template control) indicates that 1×TE buffer was used instead of DNA template. Tricine buffer was used in PCR. 0.1 μM (final concentration) of the wild-type specific beacon was used in the 50 μl reactions. TET fluorescence generated by the wild-type allele-specific beacon is shown. 0.7 pg of plasmid containing the wild-type (W/W) and 0.25 pg of plasmid containing the mutant (M/M) were mixed into 20 pg mouse gDNA as heterozygous (W/M; indicated as GT) PCR templates in 50 μl reactions. The results of reactions in which 50-100 ng of heterozygous (W/M; indicated as GT) human gDNA was used as the PCR template are as follows. Tricine buffer was used in PCR, and the reactions contained 0.2 μM (final concentration) of the wild-type specific beacon and 0.1 μM (final concentration) of the mutant specific beacon. FAM fluorescence generated by the wild-type beacon is shown. The same PCR reactions were examined for TET fluorescence generated by the mutant specific beacon. The results show that only the correspondent fluorescence (background subtracted) was detected in the homozygous DNA (i.e., FAM fluorescence in W/W and TET fluorescence in M/M). As expected, both TET and FAM fluorescence were detected in the heterozygous DNA (W/M) and no fluorescence was detected in the no template control (NTC). The intensity of the fluorescent signal detected in these samples is also as expected: higher intensity was detected in homozygous DNA and lower intensity in heterozygous DNA.

Six genotype-validated (for the Factor XIII mutation) human genomic DNA samples, provided by Cenetron Diagnostics (Austin, Tex.), were used as PCR templates (50-100 ng of DNA in 50 μl reactions). The amounts of plasmid DNA controls used were the same as described above. Tricine buffer was used in the PCR. 0.2 μM (final concentration) of the wild-type specific beacon and 0.1 μM (final concentration) of the mutant specific beacon were used. FAM fluorescence generated by the wild-type allele-specific beacon was generated and TET fluorescence generated by the mutant allele-specific was shown. The results were consistent with the predefined genotypes of these samples with 2 homozygous wild-types (W/W; indicated as GG), 2 homozygous mutants (M/M; indicated as TT) and 2 heterozygotes (W/M; indicated as GT). Fractionation of 5 μl of the amplification reaction were performed, demonstrating that the PCR templates were amplified to approximately the same extent in the reactions.

Table 12 shows results of screening of unknown genomic DNA sample for the Factor XIII mutation provided by Cenetron Diagnostics (Austin, Tex.). 50-100 ng of human genomic DNA from 50 unrelated samples was used as template in PCR (50 μl reactions) performed in tricine buffer. The reactions contained 0.2 μM (final concentration) wild-type specific beacon and 0.1 μM (final concentration) mutant specific beacon. The Factor XIII genotype, the threshold cycle values (Ct) and end-point fluorescence values for each of the 50 gDNA samples are shown in Table 12. The average Ct value for the human genotype GG (wild-type) is 24.77 (range 23.67-25.91), human genotype TT (mutant) is 26.15 (range 26.01-26.29) and human genotype GT (heterozygous) is 25.81 (range 24.59-27.06) in the FAM view (G-allele specific) and 27.82 (range 26.01-28.84) in the TET view (T-allele specific). The average end-point fluorescence values (ranges) are 2885 for GG (range), 2499 for TT (range) and 1622 (FAM, range) and 1490 for GT (TET, range). Allelic frequency for the collection of 50 human gDNA samples was G-allele=0.76 and T-allele=0.24 (similar to literature value for T-allele of 0.246, n=594 Caucasian patients (38)). Of the 50 gDNA samples, 56% (n=28) were homozygous GG, 4% (n=2) were homozygous TT and 40% (n=20) were heterozygous GT at the Factor XIII mutation. Validation of the results was performed by sequencing PCR product from three representative genotyped samples. Sequencing results are in 100% concordance with the molecular beacon allelic discrimination genotype determinations.

TABLE 12
Genotype results and Ct and end-point fluorescence
values for the human gDNA collection.

| | | G-allele | G-allele | T-allele | T-allele |
|---|---|---|---|---|---|
| Specimen | FXIIIV34L | Ct | F | Ct | F |
| 1 | GG | 24 77 | 2876 | 40 00 | 166 |
| 2 | GG | 25 14 | 2948 | 40 00 | 123 |
| 3 | GT | 25 75 | 1711 | 28 56 | 1465 |
| 4 | GG | 24 64 | 3215 | 40 00 | 6 |
| 5 | GT | 26.11 | 1406 | 28.49 | 1289 |
| 6 | GT | 25 49 | 1691 | 28 49 | 1446 |
| 7 | GT | 26.03 | 1537 | 28.32 | 1505 |
| 8 | GG | 24 37 | 2949 | 40 00 | 138 |
| 9 | GG | 24 53 | 2707 | 40 00 | 205 |
| 10 | GG | 24.54 | 2822 | 40 00 | 90 |
| 11 | GT | 25.36 | 1688 | 28 35 | 1546 |
| 12 | GT | 25 14 | 1751 | 27.85 | 1573 |
| 13 | GG | 24 23 | 2721 | 40 00 | 61 |
| 14 | GG | 24 83 | 2884 | 40 00 | 69 |
| 15 | TT | 40.00 | −35 | 26.01 | 2479 |
| 16 | GT | 26 22 | 1570 | 27 42 | 1468 |
| 17 | GT | 25 05 | 1644 | 27 01 | 1481 |
| 18 | GT | 25.67 | 1689 | 27.04 | 1540 |
| 19 | GG | 25 12 | 2805 | 40 00 | 145 |
| 20 | GG | 24 19 | 2946 | 40 00 | 90 |
| 21 | GG | 25 20 | 2828 | 40 00 | 106 |
| 22 | GG | 24.74 | 2868 | 40 00 | 164 |
| 23 | GG | 24 82 | 2809 | 40.00 | 146 |
| 24 | GG | 24 82 | 2920 | 40 00 | 114 |
| 25 | GG | 25 02 | 2778 | 40 00 | 66 |
| 26 | GG | 24 35 | 2883 | 40 00 | 95 |
| 27 | GT | 24 72 | 1773 | 27 56 | 1550 |
| 28 | GG | 24 14 | 3274 | 40 00 | −4 |
| 29 | GG | 23 67 | 2921 | 40 00 | 108 |
| 30 | GG | 23 75 | 3110 | 40 00 | 138 |
| 31 | GT | 25 51 | 1575 | 27 04 | 1607 |
| 32 | GT | 25.05 | 1588 | 27 87 | 1413 |
| 33 | GG | 24 24 | 2884 | 40.00 | 136 |
| 34 | GG | 24 24 | 2945 | 40.00 | 48 |
| 35 | GT | 25 23 | 1675 | 27 22 | 1649 |
| 36 | GT | 24 59 | 1627 | 26 41 | 1570 |
| 37 | GT | 26 77 | 1507 | 28 02 | 1393 |
| 38 | GT | 26 37 | 1614 | 28 19 | 1402 |
| 39 | GG | 25 17 | 3057 | 40.00 | 304 |
| 40 | GG | 25 21 | 3091 | 40 00 | 183 |
| 41 | GG | 25.61 | 2671 | 40 00 | 254 |
| 42 | GT | 26 69 | 1607 | 27 74 | 1537 |
| 43 | TT | 40 00 | 86 | 26 29 | 2518 |
| 44 | GT | 26.83 | 1547 | 28 29 | 1434 |
| 45 | GG | 25 64 | 2720 | 40 00 | 228 |
| 46 | GT | 27 06 | 1550 | 28 84 | 1322 |
| 47 | GG | 25 34 | 2925 | 40 00 | 313 |
| 48 | GT | 26 46 | 1682 | 27 65 | 1610 |
| 49 | GG | 25 91 | 2467 | 40.00 | 313 |
| 50 | GG | 25 34 | 2769 | 40 00 | 274 |

### EXAMPLE 8

### Target Nucleic Acid Concentration

For the mutation screening tests described, a fixed amount of human genomic DNA, 20 ng, was used. In this example, different amounts of initial target DNA are tested to determine how they affect the endpoint fluorescence and the threshold cycle (Ct) values. Both human genomic DNA (Sigma) and plasmid DNA were tested in two beacon systems (for the CCR2 and FV mutations).

In the tests, duplicated PCR reactions were carried out in the presence of different amounts of human genomic DNA (500 ng, 100 ng, 50 ng, 10 ng) or control plasmid (200 pg, 20 pg, 2 pg, 200 fg, 20 fg). The endpoint fluorescence values that were generated from 10 ng to 500 ng of genomic DNA became very similar at the last few PCR cycles. The Ct values varied from 20 to 30 with the same samples. These Ct values are well separable from those obtained in no template controls (39-40). The endpoint fluorescence values generated from 2 pg to 200 pg of plasmid DNA also became similar at the last few PCR cycles. The fluorescence values dropped when lower amounts of plasmid were used.

The effect of target DNA concentration on the endpoint fluorescence and threshold cycle values. Different amounts of human genomic DNA were used as PCR template. In both beacon systems, only the wild-type allele specific beacon produced fluorescent signal, indicating that the DNA sample is a wild-type homozygote (the mutant beacon results are not shown). Tricine buffer was used in PCR. 0.4 μM (final concentration) of the mutant specific beacon and 0.2 μM (final concentration) of the wild-type specific beacon were used. Different amounts of plasmid that contains the factor V mutation were used as PCR template. Tris buffer was used in PCR. 0.4 μM (final concentration) of the mutant specific beacon and 0.2 μM (final concentration) of the wild-type specific beacon were used. Only the mutant allele specific beacon produced fluorescent signal, as expected.

### OTHER EMBODIMENTS

Other embodiments are within the following claims.

### REFERENCES

1. Wang, D. G., et al. (1998) Science 280:1077-1082.

2. Tyagi, S. and Kramer, F. R. (1996) Nature Biotechnology 14:303-308.

3. Tyagi, S., et al.(1998) Nature Biotechnology 16:49-53.

4. Livak, K. J., et al. (1995) PCR Methods and Applications 4:357-362.

5. Kalinina, O., et al. (1997) Nucleic Acids Research 10:1999-2004.

6. TaqMan Allele Discrimination Kit (PE Applied Systems, 1998)

7. Smith, M. W., et al. (1997) Science 277:959-965.

8. Kostrikis, L. G., et al. (1998) Nature Medicine 4:350-353.

9. Liu, R., et al. (1996) Cell 86:367-377.

10. Martinson, J. J., et al. (1997) Nature Genetics 16:100-102.

11. Samson, M., et al. (1996) Nature 382:722-725.

12. Dean, M., et al. (1996) Science 273:1856-1862.

13. Winkler, C., et al. (1998) Science 279:389-393.

14. Mummidi, S., et al. (1998) Nature Medicine 4:786-793.

15. Bertina, R. M., et al. (1994) Nature 369:64-67.

16. Liu, X. Y., et al. (1995) Diagnostic Molecular Pathology 4(3): 191-197.

17. Frosst, P., et al. (1995) Nature Genetics 10:111-113.

18. Clarke, R., et al. (1991) New Engl J Med 324:1149-1155.

19. Kluijtmans, L. A. J., et al. (1995) Am J Hum Genet 58:35-41.

20. Nichols, W. C., et al. (1989) Genomics 5:535-540.

21. Wu, D. Y., et al. (1989) Proc. Natl. Acad. Sci. USA 86:2757-2760.

22. Newton, C. R., et al. (1989) Nucleic Acids Research 17:2503-2516.

23. Zhang, Y., et al. (1991) Nucleic Acids Research 19:3929-3933.

24. Chee, M., et el. (1996) Science 274:610-613.

25. Lipshutz, R. J., et al. (1995) Biotechniques 19:442-447.

26. Cronin, M. T., et al. (1996) Human Mutation 7:244-255.

27. Hacia, J. G., et al. (1996) Nature Genetics 14:441-447.

28. Yershov, K., et al. (1996) Genetics 93:4913-4918.

29. Kuppuswamy, M. N., et al. (1991) Proc. Natl. Acad. Sci. USA 88:1143-1147.

30. Landegren, U., et al. (1988) Science 241:1077-1080.

31. Haff, L. A. and Smirnov, I. P. (1997) Genome Research 378-388

32. Griffin, H. G. and Griffin, A. M.(1994) PCR Technology: Current Innovations (CRC press) p219-237.

33. Deng, H., et al. (1996) Nature 381:661-666.

34. Martin, M. P., et al. (1998) Science 282:1907-1911.

35. Jenny, R. J., et al. (1987) Proc. Natl. Acad. Sci. USA 84:4846-4850.

36. Schroder, W., et al. (1996) Haemostasis 26:233-236.

37. Kohler, H. P., et al. (1999) Thromb. Haemost. 81:511-515.

38. Kohler, H. P., et al. (1998) Thromb. Haemost. 79:8-13.

39. Wartiovaara, U., et al. (1999) Atherosclerosis 142:295-300.

40. Mikkola, H., et al. (1996) Blood 87:141-151.

41. Catto, A. J., et al. (1999) Blood 93:906-908.

1. A purified polynucleotide selected from the group consisting of SEQ ID NOS. 1-31.

2. A pair of polynucleotides for allele discrimination, said polynucleotides selected from the group consisting of SEQ ID NO. 1 and SEQ ID NO. 2; SEQ ID NO.1 and SEQ ID NO. 4; SEQ ID NO. 3 and SEQ ID NO. 2; SEQ ID NO. 3 and SEQ ID NO. 4, SEQ ID NO. 5 and SEQ ID NO. 6; SEQ ID NO. 7 and SEQ ID NO. 8; SEQ ID NO. 9 and SEQ ID NO. 10; SEQ ID NO. 11 and SEQ ID NO. 12; and SEQ ID NO. 30 and SEQ ID NO 31.

3. The pair of polynucleotides of claim 2 wherein each said pair of polynucleotides comprises first and second differentially labeled polynucleotides.

4. The pair of polynucleotides of claim 3 wherein each of said first and second differentially labeled polynucleotides comprises a pair of fluorophore/quencher labels such that said first polynucleotide comprises a first pair of fluorophore/quencher labels and said second polynucleotide comprises a second pair of fluorophore/quencher labels.

5. A pair of polynucleotide primers for a polymerase chain reaction selected from the group consisting of SEQ ID NO. 13 and SEQ ID NO. 14; SEQ ID NO. 15 and SEQ ID NO. 16; SEQ ID NO. 17 and SEQ ID NO. 18; SEQ ID NO. 19 and SEQ ID NO. 20; SEQ ID NO. 19 and SEQ ID NO. 21; SEQ ID NO. 22 and SEQ ID NO. 23; SEQ ID NO. 24 and SEQ ID NO. 25; SEQ ID NO. 26 and SEQ ID NO. 27; and SEQ ID NO. 28 and SEQ ID NO. 29.

6. A kit for allele discrimination comprising a pair of polynucleotides of claim 2 and packaging materials therefor.

7. The kit of claim 6 further comprising a pair of polynucleotide primers of claim 5 and a DNA polymerase.

8. A kit for performing a polymerase chain reaction comprising a pair of polynucleotide primers of claim 5, a DNA polymerase, and packaging materials therefor.

9. The kit of claim 6 or 8 wherein said DNA polymerase is thermostable.

10. The kit of claim 6 or 8 further comprising a buffer suitable for allele discrimination and polymerase chain reaction.

11. The kit of claim 10 further comprising a control DNA template.

12. The kit of claim 11 further comprising a DNA standard.

13. The kit of claim 12, wherein said DNA standard is genomic DNA.

14. The kit of claim 13 wherein said genomic DNA is mouse genomic DNA.

15. A kit for allele discrimination, comprising a pair of polynucleotides for allele discrimination of the CCR2 gene, wherein a first polynucleotide of said pair has the sequence presented in SEQ ID NO: 1 and a second polynucleotide of said pair of polynucleotides has the sequence presented in SEQ ID NOS: 2 or 4, a pair of polynucleotides for PCR of a region of the CCR2 gene wherein a first polynucleotide of said pair has the sequence presented in SEQ ID NO: 13 and a second polynucleotide of said pair of polynucleotides has the sequence presented in SEQ ID NO: 14, a DNA polymerase, and a buffer suitable for allele discrimination and polymerase chain reaction.

16. A kit for allele discrimination, comprising a pair of polynucleotides for allele discrimination of the CCR2 gene, wherein a first polynucleotide of said pair has the sequence presented in SEQ ID NOS: 3 and a second polynucleotide of said pair of polynucleotides has the sequence presented in SEQ ID NOS: 2 or 4, a pair of polynucleotides for PCR of a region of the CCR2 gene wherein a first polynucleotide of said pair has the sequence presented in SEQ ID NO: 13 and a second polynucleotide of said pair of polynucleotides has the sequence presented in SEQ ID NO: 14, a DNA polymerase, and a buffer suitable for allele discrimination and polymerase chain reaction.

17. A kit for allele discrimination, comprising a pair of polynucleotides for allele discrimination of the CCR5 gene, wherein a first polynucleotide of said pair has the sequence presented in SEQ ID NO: 5 and a second polynucleotide of said pair of polynucleotides has the sequence presented in SEQ ID NO: 6, a pair of polynucleotides for PCR of a region of the CCR5 gene wherein a first polynucleotide of said pair has the sequence presented in SEQ ID NO: 15 and a second polynucleotide of said pair of polynucleotides has the sequence presented in SEQ ID NO:16, a DNA polymerase, and a buffer suitable for allele discrimination and polymerase chain reaction.

18. A kit for allele discrimination, comprising a pair of polynucleotides for allele discrimination of the CCR5 gene, wherein a first polynucleotide of said pair has the sequence presented in SEQ ID NO: 5 and a second polynucleotide of said pair of polynucleotides has the sequence presented in SEQ ID NO: 6, a pair of polynucleotides for PCR of a region of the CCR5 gene wherein a first polynucleotide of said pair has the sequence presented in SEQ ID NO: 17 and a second polynucleotide of said pair of polynucleotides has the sequence presented in SEQ ID NO:18, a DNA polymerase, and a buffer suitable for allele discrimination and polymerase chain reaction.

19. A kit for allele discrimination, comprising a pair of polynucleotides for allele discrimination of the SDF1 gene, wherein a first polynucleotide of said pair has the sequence presented in SEQ ID NO: 7 and a second polynucleotide of said pair of polynucleotides has the sequence presented in SEQ ID NO: 8, a pair of polynucleotides for PCR of a region of the SDF1 gene wherein a first polynucleotide of said pair has the sequence presented in SEQ ID NO: 19 and a second polynucleotide of said pair of polynucleotides has the sequence presented in SEQ ID NOS: 20, a DNA polymerase, and a buffer suitable for allele discrimination and polymerase chain reaction.

20. A kit for allele discrimination, comprising a pair of polynucleotides for allele discrimination of the SDF1 gene, wherein a first polynucleotide of said pair has the sequence presented in SEQ ID NO: 7 and a second polynucleotide of said pair of polynucleotides has the sequence presented in SEQ ID NO: 8, a pair of polynucleotides for PCR of a region of the SDF1 gene wherein a first polynucleotide of said pair has the sequence presented in SEQ ID NO: 19 and a second polynucleotide of said pair of polynucleotides has the sequence presented in SEQ ID NOS: 21, a DNA polymerase, and a buffer suitable for allele discrimination and polymerase chain reaction.

21. A kit for allele discrimination, comprising a pair of polynucleotides for allele discrimination of the Factor V gene, wherein a first polynucleotide of said pair has the sequence presented in SEQ ID NO: 9 and a second polynucleotide of said pair of polynucleotides has the sequence presented in SEQ ID NO: 10, a pair of polynucleotides for PCR of a region of the Factor V gene wherein a first polynucleotide of said pair has the sequence presented in SEQ ID NO: 22 and a second polynucleotide of said pair of polynucleotides has the sequence presented in SEQ ID NO: 23, a DNA polymerase, and a buffer suitable for allele discrimination and polymerase chain reaction.

22. A kit for allele discrimination, comprising a pair of polynucleotides for allele discrimination of the MTHFR gene, wherein a first polynucleotide of said pair has the sequence presented in SEQ ID NO: 11 and a second polynucleotide of said pair of polynucleotides has the sequence presented in SEQ ID NO: 12, a pair of polynucleotides for PCR of a region of the MTHFR gene wherein a first polynucleotide of said pair has the sequence presented in SEQ ID NO: 24 and a second polynucleotide of said pair of polynucleotides has the sequence presented in SEQ ID NO: 25, a DNA polymerase, and a buffer suitable for allele discrimination and polymerase chain reaction.

23. A kit for allele discrimination, comprising a pair of polynucleotides for allele discrimination of the MTHFR gene, wherein a first polynucleotide of said pair has the sequence presented in SEQ ID NO: 11 and a second polynucleotide of said pair of polynucleotides has the sequence presented in SEQ ID NO: 12, a pair of polynucleotides for PCR of a region of the MTHFR gene wherein a first polynucleotide of said pair has the sequence presented in SEQ ID NO: 26 and a second polynucleotide of said pair of polynucleotides has the sequence presented in SEQ ID NO: 27, a DNA polymerase, and a buffer suitable for allele discrimination and polymerase chain reaction.

24. A kit for allele discrimination, comprising a pair of polynucleotides for allele discrimination of the Factor XIII gene, wherein a first polynucleotide of said pair has the sequence presented in SEQ ID NO: 30 and a second polynucleotide of said pair of polynucleotides has the sequence presented in SEQ ID NO: 31, a pair of polynucleotides for PCR of a region of the Factor XIII gene wherein a first polynucleotide of said pair has the sequence presented in SEQ ID NO: 28 and a second polynucleotide of said pair of polynucleotides has the sequence presented in SEQ ID NO: 29, a DNA polymerase, and a buffer suitable for allele discrimination and polymerase chain reaction.

25. The kit of claim 15-24 further comprising a control DNA template.

26. The kit of claim 15-24 further comprising a DNA standard.

27. The kit of claim 15-24, wherein said DNA standard is genomic DNA.

28. The kit of claim 27 wherein said genomic DNA is mouse genomic DNA.

29. A method for allele discrimination, comprising the steps of:
a) contacting a target nucleic acid with a pair of polynucleotides of claim 2, wherein said target nucleic acid comprises a sequence complementary to at least one polynucleotide of said pair, under conditions which permit formation of a hybrid between the target nucleic acid and said at least one polynucleotide of said pair; and
b) detecting said hybrid.

30. The method of claim 29 wherein said pair of polynucleotides is differentially labeled.

31. The method of claim 30 wherein first and second polynucleotides of said pair of polynucleotides is differentially fluorescently labeled.

32. The method of claim 29 wherein said detecting step comprises detecting emission or quenching of fluorescence.

33. A method for amplifying a target nucleic acid, comprising the steps of:
a) contacting a target nucleic acid with a pair of polynucleotide primers of claim 5, wherein said pair of primers comprises forward and reverse primers for initiating a polymerase chain reaction, under conditions which permit formation of a hybrid between said pair of polynucleotide primers and said target nucleic acid; and
b) extending the pair of polynucleotide primers in a polymerase chain reaction to form a PCR nucleic acid product that is complementary to said target nucleic acid.

34. A method for allele discrimination, comprising the steps of:
a) contacting a target nucleic acid with a pair of polynucleotides of claim 2 and a coordinate pair of polynucleotide primers of claim 5, wherein said pair of primers comprises forward and reverse primers for initiating a polymerase chain reaction on said target DNA, wherein said target nucleic acid comprises a sequence complementary to at least one polynucleotide of said pair of polynucleotides of claim 2, under conditions which permit formation of a hybrid between the target nucleic acid and said at least one polynucleotide of said pair of claim 2 and said conditions also permitting formation of a hybrid between said target nucleic acid and said pair of polynucleotides of claim 5;
b) incubating said mixture of step (a) under conditions which permit a polymerase chain reaction to generate a PCR DNA product that is complementary to said target nucleic acid and generation of loss of a signal upon formation of a hybrid between said at least one polynucleotide of said pair of claim 2 and said PCR nucleic acid product; and
c) detecting said signal or loss thereof.

35. A pair of polynucleotide probes wherein a first probe of said pair is complementary to the positive strand of a DNA duplex and a second probe of said pair is complementary to the negative strand of said DNA duplex.

36. The pair of probes of claim 35, wherein the loops of the pair of probes are non-complementary over 1 or more contiguous nucleotides.

37. The pair of probes of claim 35, wherein the stems of the pair of probes are non-complementary.
